(19) United States
(12) Patent Application Publication
(10) Pub. No.: US 2025/0387459 A1
(43) Pub. Date: Dec. 25, 2025

(54) Method of Using r-SAK Prior to Percutaneous Coronary Intervention for ST-Elevation Myocardial Infarction

(71) Applicant: Jiangsu Kanion Group Co., Ltd., Lianyungang (Jiangsu) (CN)

(72) Inventor: Chunjian Li, Nanjing (CN)

(21) Appl. No.: 19/248,955

(22) Filed: Jun. 25, 2025

Related U.S. Application Data
(60) Provisional application No. 63/663,974, filed on Jun. 25, 2024.

(51) Int. Cl. A61K 38/48; A61K 9/00; A61K 31/519; A61P 9/10

(52) U.S. Cl. CPC A61K 38/4886; A61K 9/0019; A61K 31/519; A61P 9/10; C12Y 304/24029

(57) ABSTRACT

A method of treating acute myocardial infarction in a patient by administering to the patient a combination of r-SAK and an antiplatelet agent such as ticagrelor. The acute myocardial infarction can be ST-elevation myocardial infarction. The patient can be one who is expected to receive percutaneous coronary intervention within 120 minutes from his or her first medical contact, e.g., one who is expected to receive percutaneous coronary intervention within 120 minutes from his or her first medical contact but more than 30 minutes from his or her first medical contact. A pharmaceutical composition for use in treating acute myocardial infarction in a patient is also provided which include r-SAK and an antiplatelet agent.

### BACKGROUND

Acute myocardial infarction (AMI) is a leading cause of death worldwide and accounted for more than 100 thousand deaths in the US in 2019. Among survivors of AMI, 14% to 36% develop chronic heart failure, which is associated with substantial long-term morbidity and mortality.

Primary percutaneous coronary intervention (PPCI) reduces mortality in patients with ST-elevation myocardial infarction (STEMI). Primary percutaneous coronary intervention (PCI) has been recommended as a preferred reperfusion strategy for STEMI due to its capability of reducing mortality and improving clinical outcomes. However, as the core of STEMI treatment is to minimize the total time of myocardial ischemia, guidelines recommend the use of thrombolytic therapy if PPCI is expected to be performed more than 120 minutes of presentation (“pharmacoinvasive PCI”). If percutaneous coronary intervention (PCI) cannot be performed within 120 minutes of presentation, guidelines recommend the use of thrombolytic therapy to re-open the culprit vessel as soon as possible. However, in patients who are expected to receive PPCI within 120 minutes from the first medical contact (FMC), thrombolytic therapy is not recommended. Theoretically, it would not be acceptable if PPCI is delayed for more than 60 minutes, as a preceding thrombolytic therapy to PPCI would otherwise enable early recanalization in most of the patients and improve outcome. However, previous investigations of facilitated PCI have not shown benefit and the results contradict the study hypothesis.

The unexpected results were partly due to higher stroke rates or excessive bleeding, which should be related to thrombolytic agents (Reteplase, Tenecteplase, etc), lack of optimal antithrombotic agent or inappropriate adjunctive therapies. It is uncertain whether the thrombolytic treatment in combination with contemporary antithrombotic agent would achieve good results.

### SUMMARY OF THE INVENTION

In one aspect, a method of treating AMI in a patient is provided, which comprises administering to the patient a combination of recombinant staphylokinase (r-SAK) and an antiplatelet agent prior to performing percutaneous coronary intervention on the patient.

In some embodiments, the acute myocardial infarction is ST-elevation myocardial infarction (STEMI).

In some embodiments, the patient can be one is expected (or scheduled) to receive PCI within 120 minutes from his or her FMC.

In some embodiments, the patient can be one who has STEMI symptom onset less than or equal to 12 hours and is expected to receive PCI within 120 minutes from his or her FMC.

In some embodiments, the patient is one who is expected to receive percutaneous coronary intervention within 120 minutes but more than 30 minutes from his or her FMC.

In some embodiments, the antiplatelet agent is ticagrelor.

In some embodiments, the r-SAK is administered intravenously.

In some embodiments, the r-SAK is administered in a single 5 mg bolus to the patient.

In some embodiments, the r-SAK is administered from about 10 min to about 60 min from the patient's FMC, from about 15 min to about 60 min from the patient's FMC, or from about 20 min to about 50 min from the patient's FMC, e.g., about 20 min from the FMC, about 25 min from the FMC, about 30 min from the FMC, about 35 min from the FMC, about 40 min from the FMC.

In another aspect, a pharmaceutical composition for use in treating AMI in a patient is provided, which includes r-SAK, and an antiplatelet agent. The antiplatelet agent can be ticagrelor.

### BRIEF DESCRIPTION OF THE DRAWINGS

FIG. 1. Study Flow Chart, for Example 1. r-SAK indicates recombinant staphylokinase; CMR, cardiac magnetic resonance; CAG, coronary arteriography.

FIG. 2. Primary Efficacy Endpoints in the Two Groups, for Example 1.
A, TIMI flow grade 2 to 3 and TIMI flow grade 3 60 minutes after thrombolysis analyzed by FAS (n=100 in r-SAK group and n=100 in control group).
B, TIMI flow grade 2 to 3 and TIMI flow grade 3 60 minutes after thrombolysis analyzed by PPS (n=96 in r-SAK group and n=98 in control group).
   CAG indicates coronary arteriography; FAS, full analysis set; PPS, per-protocol set; r-SAK, recombinant staphylokinase; TIMI, thrombolysis in myocardial infarction; OR, Odds ratio; CI, confidence interval. Data are presented as n (%).

FIG. 3. Subgroup Analyses of TIMI Flow by Time from Symptom to Thrombolysis and Thrombolysis to CAG, for Example 1.
A. Subgroup analyses of TIMI flow grade 2 and 3 by time from thrombolysis to CAG of ≤30 minutes, >30 to ≤60 minutes, and >60 minutes, and time from symptom to thrombolysis of ≤3 hours, >3 to ≤6 hours, and >6 hours according to FAS.
B. Subgroup analyses of TIMI flow grade 3 by time from thrombolysis to CAG of ≤30 minutes, >30 to ≤60 minutes, and >60 minutes, and time from symptom to thrombolysis of ≤3 hours, >3 to ≤6 hours, and >6 hours according to FAS.

CAG indicates coronary arteriography; TIMI, thrombolysis in myocardial infarction; FAS, full analysis set; r-SAK, recombinant staphylokinase; RD, rate difference; CI, confidence interval.

FIG. 4. Results of CMR, for Example 1.
A. CMR images. The red circles in T2 STIR series indicate edema; red circle in LGE series and r-SAK group indicates infarction area, and in LGE series and control group indicates MVO; white circle in T2 star mapping indicates IMH.
B. Infarct size detected by CMR (n=73 in r-SAK group and n=59 in control group).
C. LVEF detected by CMR (n=75 in r-SAK group and n=60 in control group).
D. Incidence of MVO detected by CMR (n=73 in r-SAK group and n=58 in control group).
E. MVO size detected by CMR (n=73 in r-SAK group and n=58 in control group).
F. Incidence of IMH detected by CMR (n=51 in r-SAK group and n=32 in control group).

CMR indicates cardiac magnetic resonance; STIR, short time inversion recovery; LGE, late gadolinium enhancement; PPS, per-protocol set; r-SAK, recombinant staphylokinase; LVEF, left ventricular ejection fraction; MVO, microvascular obstruction; INM, intramuscular hemorrhage. Data are presented as median (interquartile range), n (%), or mean±standard deviation (SD).

FIG. 5. Subgroup Analyses of CMR Detected Infarct Size by Time from Symptom to r-SAK or NS Infusion and Infarct Related Artery, for Example 1.
A. Subgroup analyses of CMR detected infarct size by time from symptom to r-SAK or NS infusion of <3 hours, ≥3 to <6 hours, and >6 hours according to PPS.
B. Subgroup analyses of CMR detected infarct size by infarct related artery of LAD, LCX and RCA according to PPS.

CMR indicates cardiac magnetic resonance; r-SAK, recombinant staphylokinase; RD, rate difference; CI, confidence interval; IRA, infarct related artery; LAD, left anterior descending artery; LCX, left circumflex artery; RCA, right coronary artery; SE, standard error; PPS per protocol set.

FIG. 6. Subgroup Analyses of TIMI Flow by Infarct Related Artery, for Example 1.
A. Subgroup analyses of TIMI flow 2 to 3 by infarct related artery of LAD, LCX and RCA according to PPS.
B. Subgroup analyses of TIMI flow 3 by infarct related artery of LAD, LCX and RCA according to PPS.

r-SAK indicates recombinant staphylokinase; IRA, infarct related artery; LAD, left anterior descending artery; LCX, left circumflex artery; RCA, right coronary artery; RD, rate difference; SE, standard error; CI, confidence interval; PPS, per protocol set.

FIG. 7. Trial design of OPTIMA-6. Patients with acute STEMI expected to undergo primary PCI ≥30 minutes and ≤120 minutes are to be randomized to a bolus of half-dose r-SAK or placebo prior to primary PCI. All recruited patients will be treated with a loading dose of aspirin 300 mg and ticagrelor 180 mg, and an intravenous bolus of unfractionated heparin (60 U/kg, and ≤5000 U) before the thrombolytic therapy.

RCT, randomized controlled trial; PCI, percutaneous coronary intervention; FMC, first medical contact; STEMI, ST-elevation myocardial infarction; r-SAK, recombinant staphylokinase

FIG. 8. OPTIMA-6 follow-up procedures. STEMI, ST-elevation myocardial infarction; r-SAK, recombinant staphylokinase; NS, normal saline; CMR, cardiac magnetic resonance

### DETAILED DESCRIPTION OF CERTAIN EMBODIMENTS

Staphylokinase (SAK) is a protein produced by Staphylococcus aureus strains that has the ability to activate plasminogen. Research indicates that SAK itself does not possess protease activity. As a plasminogen activator, SAK binds with plasminogen in a 1:1 ratio to form a complex. Under the initiation of a small amount of plasmin in the body, SAK exposes the active site of plasminogen, converting it from a single-chain to a double-chain plasmin, thus forming an active SAK-plasmin complex. This complex further activates plasminogen molecules, converting them into plasmin, thereby playing a role in dissolving blood clots. Once the thrombus is digested, the SAK-plasmin complex dissociates and is released. α2-Antiplasmin then binds to plasmin, inhibiting its activity and halting the activation of plasminogen.

Recombinant Staphylokinase (r-SAK) maintains a similar primary sequence and tertiary configuration as the native SAK produced by Staphylococcus aureus. The r-SAK gene encodes a 163 amino acid precursor protein, which includes a 27-residue signal peptide, resulting in a mature protein of 136 amino acids (Fibrinolysis, 1992, 6, 226-231). The protein is composed of a single polypeptide chain without disulfide bonds, containing two α-helices and eight β-sheets (Methods Enzymol., 1981, 80, 387). The N-terminal region is shown to be important for the activity of r-SAK (J. Biol. Chem., 1997, 272(9), 6067-6072). In terms of manufacture, high yields of biologically active r-SAK have been achieved, and production has been optimized using both prokaryotic (mainly E. coli) and eukaryotic hosts, with yeast cells also demonstrating successful high-level production. Various engineered forms of SAK with enhanced properties, such as higher fibrinolytic activity and lower antigenicity, have been developed. This includes chimeric staphylokinase variants with added antithrombotic and antiplatelet activities, as well as multifunctional SAK variants designed to improve these properties further, such as SAK-HV (hirudin) and RGD-SAK (Curr Pharm Biotechnol. 2017; 18(13):1026-1037). r-SAK is also described and may be obtained by the methods described in Chinese patent No. ZL00112273.3 and ZL00112674.1, the disclosures of which are incorporated by reference in their entireties.

A new therapeutic strategy is devised in which an SAK, e.g., a recombinant staphylokinase r-SAK, is used in combination with an antiplatelet agent, e.g., ticagrelor, for treating AMI, e.g., STEMI patients, especially for STEMI patients who are expected (or scheduled) to receive PPCI within 120 minutes from the FMC. The strategy is also herein termed OPTIMA (OPtimal management of anTIthrombotic and throMbolytic Agents). In some embodiments, the STEMI patients for the OPTIMA treatment are expected to undergo primary PCI within 120 minutes of FMC but more than 30 minutes.

In some embodiments, the r-SAK is administered at the dose of one 5 mg bolus for the patient. This is half dose of what was recommended for using r-SAK to treat AMI under the old guidelines.

In some embodiments, the antiplatelet agent, e.g., ticagrelor, is administered before r-SAK is injected intravenously to the patient prior to PCI. In some embodiments, loading doses of aspirin (oral) and an anticoagulant, e.g., intravenous heparin, can be administered together with the antiplatelet agent, or before the administration of the antiplatelet agent (and before r-SAK is injected intravenously to the patient prior to PCI). The antiplatelet agent can be administered orally.

In some embodiments, the r-SAK is administered from about 10 min to about 60 min from the patient's FMC, from about 15 min to about 60 min from the patient's FMC, or from about 20 min to about 50 min from the patient's FMC, e.g., about 20 min from the FMC, about 25 min from the FMC, about 30 min from the FMC, about 35 min from the FMC, about 40 min from the FMC. The term “about” means approximately and when use in conjunction with a value it may indicate a variation of up to 10% from the value. The other agents such as anticoagulant and/or antiplatelet agent can be administered to the patient as soon as possible after the patient's FMC, e.g., about 5 to 10 min from the patients' FMC.

In another aspect, a pharmaceutical composition comprising a combination of r-SAK and an antiplatelet agent, e.g., ticagrelor, is provided. r-SAK and the antiplatelet agent can be provided in the same package or different packages, and/or in separate dosage forms. The term “pharmaceutical composition” includes preparations suitable for administration to a patient, in which the active ingredients such as r-SAK and antiplatelet agent can be included in combination with a pharmaceutically acceptable carrier, or optionally two or more pharmaceutically acceptable carriers. The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Typically, pharmaceutically acceptable carriers are sterilized and/or substantially pyrogen-free.

### Abbreviations and Acronyms Used in the Examples

ACT activated clotting time
AMI acute myocardial infarction
CABG coronary artery bypass grafting
CAG coronary arteriography
CMR cardiac magnetic resonance
CTFC corrected TIMI frame count
BARC Bleeding Academic Research Consortium
DSA digital subtraction angiography
DICOM digital imaging and communications in medicine
DSMC data and safety monitoring committee
ERC event review committee
EDC electronic data capture
FAS full analysis set
GPI platelet glycoprotein IIb/IIIa receptor inhibitor
IQR inter quartile range
IRA infarct related artery
IMH intramuscular hemorrhage
ITT intention-to-treat
LMWH low molecular weight heparin
LVEF left ventricular ejection fraction
MACE major adverse cardiovascular events
MVO microvascular obstruction
NS normal saline
NT-proBNP N-terminal pro-B-type natriuretic peptide
PCI percutaneous coronary intervention
PPCI primary percutaneous coronary intervention
PPS per-protocol set
r-SAK recombinant staphylokinase
SC steering committee
SS safety set
STEMI ST-elevation myocardial infarction
TMPFC TIMI myocardial perfusion frame count
TIA transient ischemic attack
TIMI thrombolysis in myocardial infarction

### Example 1: Randomized Study Evaluation of r-SAK Administration (OPTIMA-5) Executive Summary

An open-label, prospective, multicenter, randomized study was performed, which is also referred to OPTIMA-5 herein. Patients aged 18 to 75 who were within 12 hours of symptom onset of STEMI and expected to undergo PCI within 120 minutes were enrolled. Patients were administered loading doses of aspirin and ticagrelor orally and intravenous heparin and were randomized to receive 5 mg bolus of r-SAK or normal saline (NS) intravenously prior to PCI. The primary endpoint was thrombolysis in myocardial infarction (TIMI) flow grade 2 to 3 or grade 3 in the infarct related artery (IRA) 60 minutes after thrombolysis. The infarct size was detected by cardiac magnetic resonance (CMR) 5 days after randomization. The safety endpoint was major bleeding (BARC ≥3) during 30-day follow-up.

A total of 283 patients were screened from 8 centers, and 200 were randomized (median age 58.5 years, 14% female). The median symptom to thrombolysis time was 252.5 (IQR 142.8 to 423.8) minutes and thrombolysis to coronary arteriography was 50.0 (IQR 37.0 to 66.0) minutes. Patients randomized to r-SAK compared with NS more often had TIMI flow grade 2 to 3 (69.0% vs. 29.0%; P<0.001) and TIMI flow grade 3 (51.0% vs. 18.0%; P<0.001) and had smaller infarct size (21.91±10.84% vs. 26.85±12.37%; P=0.016). There was no increase in major bleeding (r-SAK 1.0% vs. control 3.0%; P=0.616).

In sum, the efficacy of adjunctive thrombolysis with r-SAK for patients with STEMI undergoing PPCI within 120 minutes of presentation was investigated. It was found that a single bolus half-dose r-SAK intravenously prior to timely PPCI for STEMI improves IRA patency and reduces infarct size without increasing major bleeding.

The objective of OPTIMA-5 trial was to determine whether in patients presenting with STEMI a single bolus of half-dose r-SAK in combination with contemporary antiplatelet agent (ticagrelor) prior to timely PCI leads to improved patency of the IRA and reduces the infarct size, and thereby potentially improves immediate and long-term clinical outcome.

### Methods

### Study Design

The OPTIMA-5 trial is an investigator-initiated, prospective, multi-center, randomized, controlled trial comparing a single bolus of half-dose r-SAK with NS in patients with STEMI presenting ≤12 hours of symptom onset and expected to undergo PCI within 120 minutes.

Between Oct. 29, 2021 and Aug. 14, 2022, 283 STEMI patients were screened in 8 centers in China and 200 were randomized to r-SAK group or control in a 1:1 ratio using a computer-generated randomization sequence. The randomization sequence was maintained by an independent third party not involved in any other aspect of the trial. Investigators randomized patients by calling the third party who confirmed eligibility before providing information regarding treatment allocation.

The study drug [r-SAK, 5 mg (250,000 AU), Batch number: S20040074)] was provided by Kanion Pharmaceutical Group, Jiangsu, China.

This study was performed in accordance with the Declaration of Helsinki of Good Clinical Practice and was approved by the Research Ethics Board of the First Affiliated Hospital of Nanjing Medical University (2021-SR-309). Written informed consent was obtained from each participant.

### Eligibility Criteria

Patients were eligible for inclusion based on the following criteria: 1) age 18-75 years; 2) presenting with STEMI according to the universal definition of AMI;¹⁴ 3) time from onset of persistent chest pain to randomization <12 hours; 4) PPCI expected to be performed within 120 minutes.

Patients were excluded if they met any of the following criteria: 1) cardiogenic shock; 2) active bleeding or at high risk of bleeding (including grade III or IV retinopathy or retinal gastrointestinal or urinary tract hemorrhage within the past 1 month); 3) ischemic stroke or transient ischemic attack (TIA) in the past 6 months; 4) history of hemorrhagic stroke; 5) platelet count <100×10⁹/L or hemoglobin <100 g/L; 6) known intracranial aneurysm; 7) severe trauma, surgery or head injury (within 1 month); 8) suspected aortic dissection or infective endocarditis; 9) recent puncture with difficult hemostasis by compression (e.g., visceral biopsy, compartment puncture); 10) currently taking anticoagulants; 11) poorly controlled hypertension (≥180/110 mmHg); 12) hepatic or renal impairment (glutamic-pyruvic transaminase, glutamic oxalacetic transaminase, or γ-glutamyl transferase >2.5 times upper limit of normal value; creatinine >1.5 times upper limit of normal value); 13) known allergy to r-SAK; 14) pregnancy, lactation, or planning for pregnancy; 15) history of myocardial infarction or coronary artery bypass grafting (CABG); 16) having taken antiplatelet drugs other than aspirin and ticagrelor, such as clopidogrel, prasugrel or cilostazol after the symptom onset; 17) patients with other conditions that made them unsuitable to be recruited at the discretion of the investigators.

### Thrombolytic and Medical Therapy

Eligible patients were randomly assigned to r-SAK or control. All the patients were treated with aspirin 300 mg and ticagrelor 180 mg, and an intravenous bolus of unfractionated heparin (60 U/kg, and ≤5000 U) after being recruited. Patients in r-SAK group were given an intravenous bolus of 5 mg r-SAK over 3 minutes and those in control group were given 10 ml NS over the same period. Patients were blinded to treatment allocation.

The protocol did not allow use of platelet glycoprotein IIb/IIIa receptor inhibitor (GPI) or low molecular weight heparin (LMWH) unless there was evidence of heavy thrombus burden at coronary arteriography (CAG). The protocol recommended that all patients receive background medical therapy according to guideline recommendations.^{7,15}

### Coronary Intervention

The protocol required measurement of the activated clotting time (ACT) after sheath insertion and tailoring of the dose of unfractionated heparin to achieve a value between 200 and 250 seconds.

For assessment of thrombolysis in myocardial infarction (TIMI) flow, CAG was set at 15 frames per second (normal). The duration of angiography at each position was ≥5 cardiac cycles (or until contrast agent was fully drained). The following projections were required: if the IRA was the left coronary artery: 1) RAO 30°, CAU 30°; 2) RAO 30°, CRA 30°; if the IRA was the right coronary artery: 1) LAO 40°, CRA 0°; 2) LAO 30°, CRA 30°.

If initial CAG was performed within 60 minutes of r-SAK or NS infusion and the TIMI flow in the IRA was grade 0-2, immediate PCI was to be performed. If TIMI flow was grade 3, CAG was to be repeated in the same setting at 60 minutes after r-SAK or NS infusion. If CAG was performed 60 minutes after r-SAK or NS infusion, and the TIMI flow was grade 0-2, immediate PCI was to be performed, while if TIMI flow was grade 3, further revascularization strategy was at the discretion of the attending physicians.

All CAG images were digitally stored in digital imaging and communications in medicine (DICOM) format and labelled with the center and patient ID numbers for subsequent offline analyses by two event review committee (ERC) members blinded to the treatment strategy and all other clinical information. Any disagreements were resolved by a third reviewer.

### Cardiac Magnetic Resonance

Assessment of infarct size, microvascular obstruction (MVO), left ventricular ejection fraction (LVEF) and intramyocardial hemorrhage (IMH) was performed by cardiac magnetic resonance (CMR) scanning (3.0 tesla) 5 days after randomization (Philips, Ingenia, the Netherlands; Siemens, Erlangen, Germany; and GE Healthcare, Milwaukee, USA) using the same acquisition protocol. The imaging sequence utilized to measure IMH was dark-blood T2 weighted imaging: Short T1 Inversion Recovery (STIR) sequence, capturing 10-12 layers of SAX; TR/TE, 2 ms/75 ms; reverse angle, 90°; layer thickness, 8 mm; FOV, 300×300 mm; Matrix, 160×139 mm; the spatial resolution, 1.3×1.65×8 mm³.

CMR data were recorded in DICOM format for subsequent analyses by an independent core laboratory blinded to treatment allocation and all other clinical information.

### Study Endpoints

The primary endpoints were the percentage of TIMI flow grade 2 to 3, and TIMI flow grade 3 in the IRA after 60 minutes of the r-SAK or NS infusion. The secondary endpoints included the occurrence of slow-reflow or no-reflow during PCI, and infarct size, etc as shown in Table AS1.

TABLE AS1
Secondary Endpoints of the Study.
Secondary endpoints

| | |
|---|---|
| 1) | The occurrence of slow or no reflow during CAG or PCI; |
| 2) | The percentage of TIMI flow grade 3 after PCI; |
| 3) | CTFC and TMPFC after PCI; |
| 4) | Infarct size, MVO, LVEF and IMH, detected by CMR at 5^{th} day after AMI; |
| 5) | MACCEs, defined as composite of all-cause death, myocardial infarction, unplanned |
| | revascularization, ischemic stroke and cardiogenic re-hospitalization recorded during |
| | 30-day follow-up; |
| 6) | Major bleeding (BARC ≥3) and minor bleeding (BARC ≤2) events during 30-day |
| | follow-up; |
| 7) | Clinical net benefits of MACCEs and major bleeding events within 30 days; |
| 8) | Malignant arrhythmia after thrombolysis and during hospitalization. |

CAG indicates coronary arteriography; PCI, percutaneous coronary intervention; TIMI, thrombolysis in myocardial infarction; CTFC, corrected TIMI frame count; TMPFC, TIMI myocardial perfusion frame count; LVEF, left ventricular ejection fraction; MVO, microvascular obstruction; IMH, intramuscular hemorrhage; CMR, cardiac magnetic resonance; AMI, acute myocardial infarction; MACCEs, major adverse cardiovascular and cerebrovascular events; BARC, bleeding academic research consortium.
The safety endpoint was the incidence of major bleeding defined as Bleeding Academic Research Consortium (BARC) ≥3 during 30-day follow-up.¹⁷

### Statistical Analysis

The sample size is driven by the primary efficacy estimation. Based on the data of previous studies, the excepted reperfusion rate of TIMI flow grade 2 to 3 is estimated to be 60% in the r-SAK group and 30% in the control group, and the expected reperfusion rate of TIMI flow grade 3 would be 55% and 25%, respectively. A total of 150 subjects (75 subjects per group) 20 would yield at least 95% power to detect the difference of primary efficacy endpoints between two arms at the 2-sided level of significance of 0.05, and 188 subjects would be required allowing for 20% of randomized patients who do not undergo angiography, we determined a final sample size of 200 patients.

The primary, safety and secondary endpoints were analyzed using the full analysis set (FAS) based on intention-to-treat (ITT) criteria, safety set (SS) or per-protocol set (PPS) as appropriate, by a statistician blinded to the treatment allocation and all other clinical data. Continuous data were presented as means and standard deviation or medians and interquartile ranges. The Kolmogorov-Smirnov test was used to evaluate whether the continuous variables were normally distributed. The significance of any differences in continuous variables between the two groups was determined by Student's t-test or Mann-Whitney U-test. Categorical variables were presented as number and percentages and the significance of any differences were evaluated using a χ² or Fisher's exact tests, as appropriate. The fixed sequential stepwise downward method was adopted to control the type I error for the two primary analyses. Analyses of TIMI flow and infarct size were performed in subgroups defined by r-SAK or NS infusion to CAG time (<30 minutes; ≥30 to <60 minutes; ≥60 minutes) and symptom to r-SAK or NS infusion time (<3 hours; ≥3 to <6 hours; ≥6 hours). All tests were two-sided and a P-value of <0.05 was considered statistically significant. All analyses were performed using SAS version 9.4 (SAS Institute, Cary, NC, USA).

If a patient could not accept CAG due to any reason, the primary endpoint of the patient would be considered as TIMI flow grade 0. If a patient underwent PCI within 60 minutes, the TIMI flow grade at initial CAG was considered as primary endpoint.

### Results

### Patient Characteristics

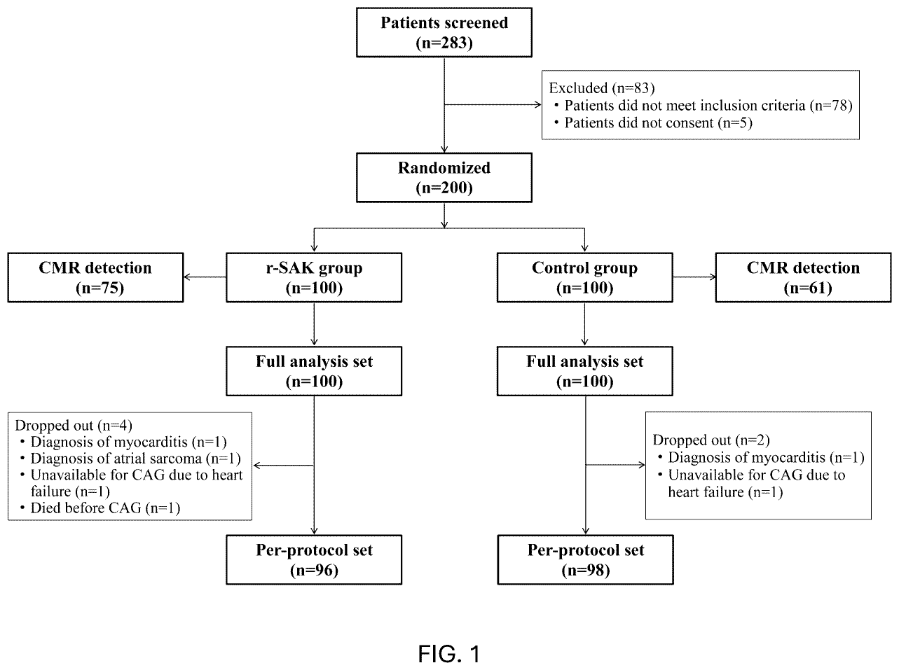

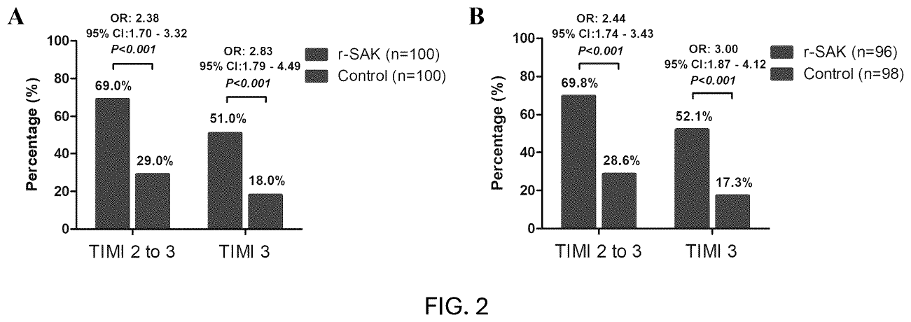

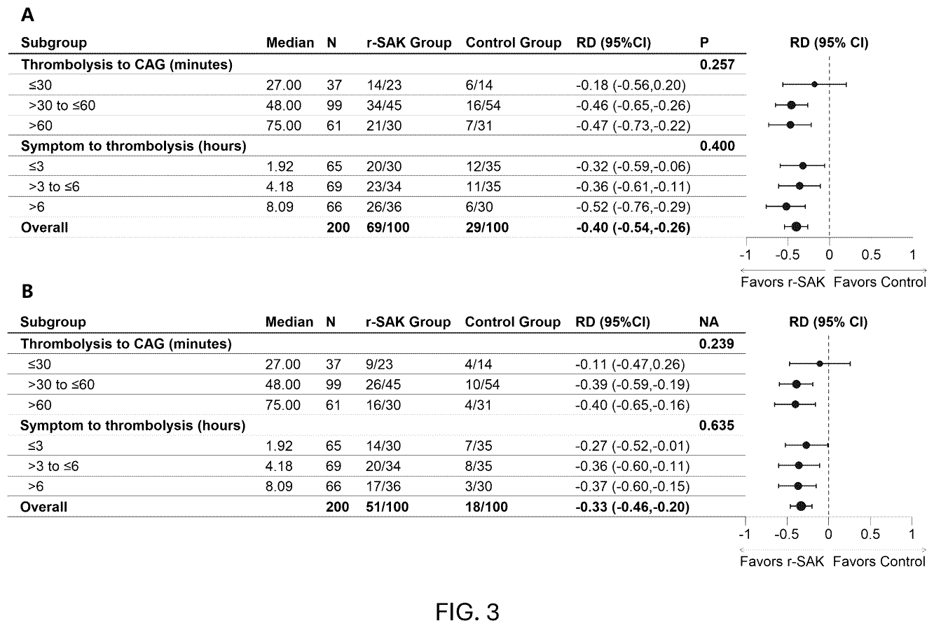

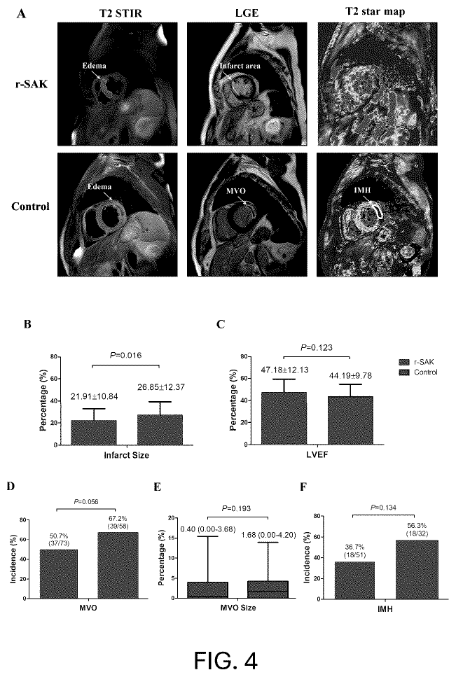

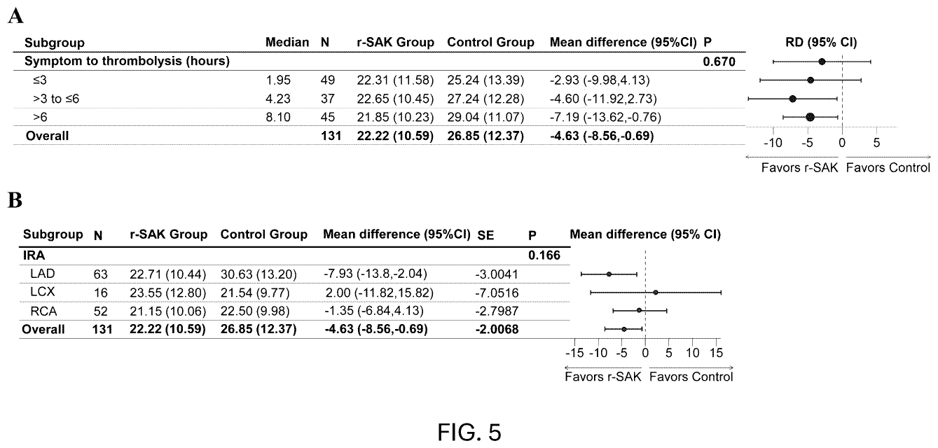

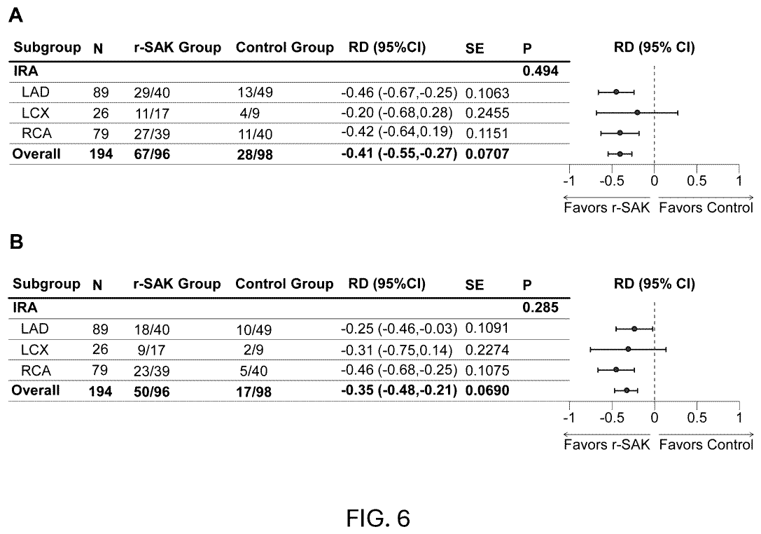

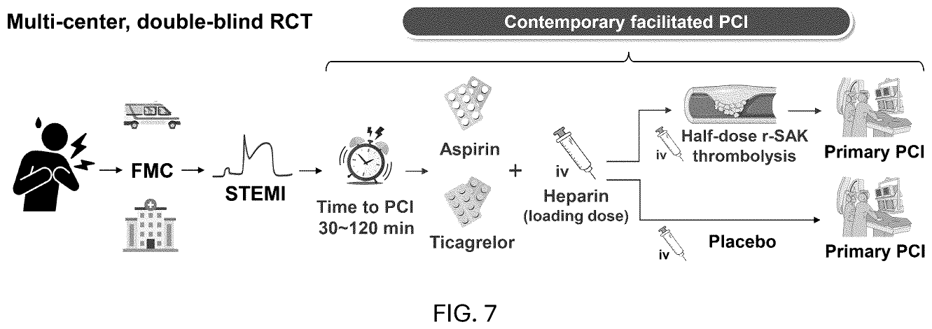

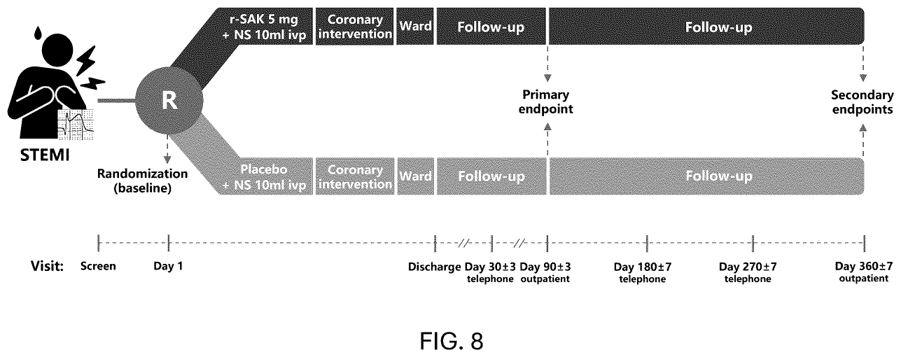

The flowchart of the trial is shown in FIG. 1. Between Oct. 29, 2021 and Aug. 14, 2022, a total of 283 STEMI patients were screened at 8 centers in China and 200 patients randomly assigned to receive r-SAK (n=100) or control (n=100). All patients were included in the ITT analysis. After randomization, 2 patients were diagnosed with myocarditis and 1 with atrial sarcoma, 2 patients did not undergo CAG due to heart failure, and 1 patient died of cardiac rupture before CAG. The remaining 194 (96 in r-SAK group and 98 in control group) patients were included in the per protocol analysis.

Participants' baseline characteristics are presented in Table A1. The median age was 58.5 years, 14.0% were female, 54.0% had hypertension, 27.0% diabetes mellitus, 13.0% hypercholesterolemia, and 58.0% were current smokers. There were no significant differences in baseline characteristics between the two groups.

TABLE A1
Baseline and Coronary Intervention Related
Characteristics of the Recruited Patients.

| | r-SAK Group | | Control Group | |
|---|---|---|---|---|
| Characteristics | (N = 100) | | (N = 100) | |
| Age (IQR) - yr | 56.0 | (50.0-65.0) | 60.0 | (50.8-66.3) |
| Male sex - no. (%) | 86 | (86.0) | 86 | (86.0) |
| BMI (IQR) - kg/m² | 25.4 | (23.4-27.5) | 24.2 | (22.5-27.2) |
| Systolic blood pressure (±SD) - mmHg | 130.5 ± 22.3 | | 123.9 ± 25.0 | |
| Diastolic blood pressure (±SD) - mmHg | 81.5 ± 14.8 | | 76.3 ± 16.8 | |
| Heart rate (IQR) - beats/min | 77.5 | (72.0-89.3) | 76.0 | (70.0-88.0) |
| Killip class - no. (%) | | | | |
| I | 93 | (93.0) | 92 | (92.0) |
| II | 6 | (6.0) | 7 | (7.0) |
| III | 1 | (1.0) | 1 | (1.0) |
| IV | 0 | (0) | 0 | (0) |
| Previous disease history - no. (%) | | | | |
| Hypertension | 60 | (60.0) | 48 | (48.0) |
| Diabetes mellitus | 28 | (28.0) | 26 | (26.0) |
| Hypercholesterolemia | 12 | (12.0) | 14 | (14.0) |
| Ischemic stroke | 5 | (5.0) | 7 | (7.0) |
| Peripheral artery disease | 1 | (1.0) | 0 | (0.0) |
| Coronary artery intervention | 9 | (9.0) | 6 | (6.0) |
| Family history of CAD - no. (%) | 5 | (5.0) | 2 | (2.0) |
| Current smoker- no. (%) | 63 | (63.0) | 53 | (53.0) |
| Time delay (IQR) - minutes | | | | |
| Symptom to door | 209.5 | (120.0-366.5) | 206.5 | (94.3-373.3) |
| Symptom to thrombolysis | 254.5 | (148.8-428.3) | 245.5 | (137.5-399.0) |
| Thrombolysis to CAG† | 49.0 | (32.0-66.0) | 52.0 | (38.5-65.0) |
| Radial artery access - no. (%)* | 94 | (97.9) | 93 | (94.9) |
| Infarct-related artery - no. (%)* | | | | |
| LM | 0 | (0.0) | 0 | (0.0) |
| LAD | 40 | (41.7) | 49 | (50.0) |
| LCX | 17 | (17.7) | 9 | (9.2) |
| RCA | 39 | (40.6) | 40 | (40.8) |
| Treatment options - no. (%)* | | | | |
| PCI | 87 | (90.1) | 93 | (94.9) |
| CABG | 3 | (3.1) | 3 | (3.1) |
| PCI rejected by patients | 2 | (2.1) | 1 | (1.0) |
| No severe lesion to treat | 4 | (4.2) | 1 | (1.0) |
| Thrombus aspiration - no. (%)* | 9 | (9.3) | 20 | (20.4) |
| GPI use - no. (%)* | 22 | (22.9) | 30 | (30.6) |
| Stents implanted in IRA (IQR)* | 1 | (1-1) | 1 | (1-2) |

†1 patient died before CAG in r-SAK group, 2 patients were unavailable for CAG due to heart failure and 1 in each group. As a result, 98 patients in r-SAK group and 99 in control received CAG.
*Analyzed by per-protocol set (n = 96 in r-SAK group and n = 98 in control group).
r-SAK indicates recombinant staphylokinase; BMI, Body mass index; CAD, coronary artery disease; CAG, coronary arteriography; LM, left main artery; LAD, left anterior descending artery; LCX, left circumflex artery; RCA, right coronary artery; PCI, percutaneous coronary intervention; CABG, coronary artery bypass grafting; GPI, Platelet glycoprotein IIb/IIIa receptor inhibitor; IRA, infarct-related artery; PTCA, Percutaneous transluminal coronary angioplasty. Data are presented as median (interquartile range), n (%), or mean ± SD.

The median time from symptom onset to presentation was 209.5 minutes in r-SAK group and 206.5 minutes in control group; the median time from symptom onset to r-SAK or NS infusion was 254.5 minutes in r-SAK group and 245.5 minutes in control group; and the median time from r-SAK or NS infusion to CAG was 49.0 minutes in r-SAK group and 52.0 minutes in control group.

Coronary intervention related characteristics of the patients are shown in Table A1. 92.8% of the patients (87 in r-SAK group and 93 in control group) received PPCI. All the coronary intervention related characteristics in the two groups were comparable. However, there was a trend of more thrombus aspiration, more GPI infusion and more stent implantation in the IRA in the control group.

Thrombolytic Efficacy of r-SAK

Within 60 minutes of the r-SAK or NS infusion, CAGs were performed in 129 (66 in r-SAK group; 63 in NS group) of the recruited patients, among whom 42 (32 in r-SAK group, 10 in NS group) reached TIMI flow grade 3 in IRA at initial CAG, and received another CAG at 60 minutes (TIMI flow grade 3 maintained in all these 42 patients). Their TIMI flow grade at 60 minutes was considered as the primary endpoint. Another 5 (3 in r-SAK group, 2 in NS group) reached TIMI flow grade 3 at initial CAG, but received PCI immediately. Their initial TIMI flow grade was considered as the primary endpoint. The last 82 patients (31 in r-SAK group, 51 in NS group) had TIMI flow less than grade 3 in IRA at initial CAG and received PCI immediately, their initial TIMI flow grade was considered as the primary endpoint. 68 patients (32 in r-SAK group, 36 in NS group) underwent CAG after 60 minutes of the r-SAK or NS infusion, and their TIMI flow grade at initial CAG was considered as primary endpoint.

After 60 minutes of the r-SAK or NS infusion, patients randomized to r-SAK compared with NS more often had TIMI flow grade 2 to 3 (69.0% vs. 29%; P<0.001) (FIG. 2A) and TIMI flow grade 3 (51.0% vs. 18.0%; P<0.001) (FIG. 2B). Results were similar in the per protocol analysis: patients randomized to r-SAK compared with NS more often had TIMI flow grade 2 to 3 (69.8% vs. 28.6%; P<0.001) (FIG. 2C) and TIMI flow grade 3 (52.1% vs. 17.3%; P<0.001) (FIG. 2D).

Subgroup analyses showed completely consistent effects on both TIMI flow 2 to 3 and TIMI flow 3 of IRA irrespective of time intervals of r-SAK or NS infusion to CAG and symptom to r-SAK or NS infusion (FIG. 3). Similarly, consistent effects were found regarding different culprit vessels (FIG. 6).

The incidence of slow-reflow and no-reflow was 17.7% in r-SAK group compared with 32.7% in control group during PCI (P=0.026) (Table AS2). The percentage of TIMI flow grade 3 post PCI was 85.4% in r-SAK group compared with 78.6% in control group (P=0.292) (Table AS2).

TABLE AS2
Results of Secondary Endpoints in the Two Groups.

| | r-SAK Group | | Control Group | | |
|---|---|---|---|---|---|
| | (N = 100) | | (N = 100) | | P Value |
| Slow or no reflow during | 17 | (17.7) | 32 | (32.7) | 0.026 |
| CAG or PCI - no. (%) * | | | | | |
| TIMI flow grade 3 | 82 | (85.4) | 77 | (78.6) | 0.292 |
| after PCI - no. (%) * | | | | | |
| CTFC (IQR) - frame † | 26.0 | (19.0-36.0) | 26.0 | (18.3-36.0) | 0.626 |
| TMPFC (IQR) - frame ^{††} | 104.0 | (82.0-127.0) | 108.0 | (82.0-172.0) | 0.596 |
| Infarct size (±SD) - % # | 21.9 ± 10.8 | | 26.9 ± 12.4 | | 0.016 |
| LVEF (±SD) - % ## | 47.2 ± 12.1 | | 44.2 ± 9.8 | | 0.123 |
| MVO - no. (%) ¶ | 37 | (50.7) | 39 | (67.2) | 0.056 |
| MVO size (IQR) - % ¶ | 0.40 | (0.00-3.68) | 1.68 | (0.00-4.20) | 0.193 |
| IMH - no. (%) ¶¶ | 18 | (36.7) | 18 | (56.3) | 0.134 |

* Analyzed by per-protocol set (n = 96 in r-SAK group and n = 98 in control group).
† n = 94 in r-SAK group and n = 95 in control group.
^{††} n = 51 in r-SAK group and n = 52 in control group.
Some do not have CTFC and TMPFC results because they did not have enough duration of angiography, or the contrast agent was not completely emptied.
# n = 73 in r-SAK group and n = 59 in control group.
## n = 75 in r-SAK group and n = 60 in control group.
¶ n = 73 in r-SAK group and n = 58 in control group.
¶¶ n = 51 in r-SAK group and n = 32 in control group.
Some do not have results of infarct size, LVEF, MVO and IMH due to poor quality of images detected by CMR.
r-SAK indicates recombinant staphylokinase; CAG, coronary arteriography; PCI, percutaneous coronary intervention; TIMI, thrombolysis in myocardial infarction; CTFC, corrected TIMI frame count; TMPFC, TIMI myocardial perfusion frame count; LVEF, left ventricular ejection fraction; MVO, microvascular obstruction; IMH, intramuscular hemorrhage; CMR, cardiac magnetic resonance. Data are presented as median (interquartile range), n (%), or mean ± SD.

Regarding the myocardial reperfusion markers, the CTFC [r-SAK: 26.0 (interquartile range, 19.0 to 36.0) vs. Control: 26.0 (interquartile range, 18.3 to 36.0); P=0.626, Table AS2] and TMPFC [r-SAK: 104.0 (interquartile range, 82.0 to 127.0) vs. Control: 108.0 (interquartile range, 82.0 to 172.0); P=0.596, Table AS2] showed no significant differences between the 2 groups.

### Results of CMR

A total of 136 (68.0%) patients underwent in-hospital CMR scan (FIG. 4A). Comparing the baseline and coronary intervention related characteristics of the patients who received CMR scan and those who did not, patient who received CMR scan tended to be more from r-SAK group, with better Killip class, less having previous PCI, and less being transferred to the surgery (Table AS3).

TABLE AS3
Baseline and Coronary Intervention Related Characteristics of
the Patients Who Received CMR Scan and Those Who Did Not.

| | CMR | | No CMR | | P |
|---|---|---|---|---|---|
| Characteristics | (N = 136) | | (N = 64) | | Value |
| r-SAK group - no. (%) | 75 | (55.1) | 25 | (39.1) | 0.034 |
| Age (IQR) - yr | 57.5 | (50.0-65.0) | 61.0 | (51.3-68.8) | 0.052 |
| Male sex - no. (%) | 118 | (86.8) | 54 | (84.4) | 0.814 |
| BMI (IQR) - kg/m² | 24.8 | (22.9-27.4) | 24.7 | (22.9-27.4) | 0.637 |
| Systolic blood | 128.8 ± 24.5 | | 123.8 ± 22.3 | | 0.172 |
| pressure (±SD) - mmHg | | | | | |
| Diastolic blood | 79.6 ± 15.4 | | 77.5 ± 17.2 | | 0.378 |
| pressure (±SD) - mmHg | | | | | |
| Heart rate (IQR) - beats/min | 76.0 | (72.0-85.2) | 77.0 | (70.0-93.0) | 0.689 |
| Killip class - no. (%) | | | | | 0.015 |
| I | 130 | (95.6) | 55 | (85.9) | |
| II | 6 | (4.4) | 7 | (10.9) | |
| III | 0 | (0.0) | 2 | (3.1) | |
| IV | 0 | (0.0) | 0 | (0.0) | |
| Previous disease history - no. (%) | | | | | |
| Hypertension | 70 | (51.5) | 38 | (59.4) | 0.371 |
| Diabetes mellitus | 39 | (28.7) | 15 | (23.4) | 0.543 |
| Hypercholesterolemia | 17 | (12.5) | 9 | (14.1) | 0.935 |
| Ischemic stroke | 6 | (4.4) | 6 | (9.4) | 0.289 |
| Peripheral artery disease | 1 | (0.7) | 0 | (0.0) | 1.000 |
| Coronary artery intervention | 6 | (4.4) | 9 | (14.1) | 0.033 |
| Family history of CAD - no. (%) | 7 | (5.1) | 0 | (0.0) | 0.099 |
| Current smoker- no. (%) | 77 | (56.6) | 39 | (60.9) | 0.824 |
| Time delay (IQR) - minutes | | | | | |
| Symptom to door | 206.0 | (97.3-366.5) | 211.0 | (135.5-373.3) | 0.785 |
| Symptom to thrombolysis | 252.0 | (133.8-425.5) | 253.0 | (187.3-397.5) | 0.867 |
| Thrombolysis to CAG† | 50.0 | (35.8-64.0) | 52.0 | (40.0-69.0) | 0.205 |
| Radial artery access - no. (%)† | 132 | (97.1) | 58 | (95.1) | 0.425 |
| Infarct-related artery - no. (%)* | | | | | 0.625 |
| LM | 0 | (0.0) | 0 | (0.0) | — |
| LAD | 65 | (47.8) | 26 | (41.3) | |
| LCX | 16 | (11.8) | 10 | (15.9) | |
| RCA | 54 | (39.7) | 25 | (39.7) | |
| None# | 1 | (0.7) | 2 | (3.2) | |
| Treatment options - no. (%)* | | | | | <0.001 |
| PCI | 132 | (97.1) | 50 | (79.4) | |
| CABG | 0 | (0.0) | 6 | (9.5) | |
| PCI rejected by patients | 1 | (0.7) | 2 | (3.2) | |
| No severe lesion to treat | 3 | (2.2) | 5 | (7.9) | |

†1 patient died before CAG and 2 patients were unavailable for CAG due to heart failure in the No CMR group, n = 61.
*1 patient died before CAG in the No CMR group, n = 63.
#1 patient was diagnosed as myocarditis in CMR group. 2 patients were diagnosed as myocarditis and atrial sarcoma respectively in No CMR group. Therefore, 3 patients had no infarct-related artery.
CMR indicates cardiac magnetic resonance; r-SAK, recombinant staphylokinase; BMI, Body mass index; CAD, coronary artery disease; CAG, coronary arteriography; LM, left main artery; LAD, left anterior descending artery; LCX, left circumflex artery; RCA, right coronary artery; PCI, percutaneous coronary intervention; CABG, coronary artery bypass grafting. Data are presented as median (interquartile range), n (%), or mean ± SD.

Compared with those who received NS, patients randomized to receive r-SAK had smaller infarct size (21.91±10.84% vs. 26.85±12.37%; P=0.016, FIG. 4B, Table AS2), though no significant difference was found regarding LVEF (47.18±12.13% vs. 44.19±9.78%; P=0.123, FIG. 4C, Table AS2), ratio of microvascular obstruction (50.7% vs. 67.2%; P=0.056, FIG. 4D, Table AS2), area of MVO [0.40 (interquartile range, 0.00 to 3.68) vs. 1.68 (interquartile range, 0.00 to 4.20); P=0.193, FIG. 4E, Table AS2)], or incidence of intramuscular hemorrhage (36.7% vs. 56.3%; P=0.134, FIG. 4F, Table AS2).

Subgroup analyses showed consistent effects on infarct size irrespective of time from symptom onset to r-SAK or NS infusion (FIG. 5A). Another subgroup analyses showed significant reduction of infarct size in r-SAK group compared with the control when the culprit vessel was LAD, while no significant reduction was found when the culprit vessel was LCX or RCA (FIG. 5B).

### Clinical Outcome

The clinical outcomes at 30-day follow-up are summarized in Table A2.

TABLE A2
Clinical Events of the Recruited Patients in the Two Groups.

| | r-SAK Group | | Control Group | | |
|---|---|---|---|---|---|
| | (N = 100) | | (N = 100) | | P Value |
| MACCEs | 3 | (3.0) | 6 | (6.0) | 0.498 |
| Death | 2 | (2.0) | 2 | (2.0) | — |
| Ischemic stroke | 1 | (1.0) | 2 | (2.0) | — |
| Reinfarction | 0 | (0.0) | 1 | (1.0) | — |
| Cardiogenic re-hospitalization | 0 | (0.0) | 1 | (1.0) | — |
| Major bleeding - no. (%) | | | | | 0.246 |
| BARC 3 | 0 | (0.0) | 2 | (2.0) | — |
| BARC 4 | 0 | (0.0) | 1 | (1.0) | — |
| BARC 5 | 0 | (0.0) | 0 | (0.0) | — |
| Minor bleeding - no. (%) | | | | | 0.611 |
| BARC 1 | 21 | (21.0) | 20 | (20.0) | — |
| BARC 2 | 3 | (3.0) | 1 | (1.0) | — |
| Reperfusion injury - no. (%) | | | | | |
| Malignant arrhythmias | 9 | (9.0) | 18 | (18.0) | 0.098 |
| Transient hypotension | 7 | (7.0) | 4 | (4.0) | 0.535 |
| Allergic reaction - no. (%) | 0 | (0.0) | 0 | (0.0) | — |

r-SAK indicates recombinant staphylokinase; MACCEs, Major Adverse Cardiovascular and Cerebrovascular Events; BARC, bleeding academic research consortium. Data are presented as n (%).

4 patients died during the 30-day follow-up, 2 (2.0%) in r-SAK group and 2 (2.0%) in control group. The causes of death were cardiac rupture and cardiac shock in r-SAK group, and 2 patients with cardiac shock in the control group. There was no significant difference in major adverse cardiovascular and cerebrovascular events between the r-SAK and control groups (3.0% vs. 6.0%; P=0.498) (Table A2).

No significant differences were found regarding the incidences of the major bleeding (1.0% vs. 3.0%; P=0.616), minor bleeding (24.0% vs. 21.0%; P=0.611), reperfusion induced malignant arrhythmias (9.0% vs. 18.0%; P=0.098) or transient hypotension (7.0% vs. 4.0%; P=0.535). No patients experienced allergic reaction in either group.

There were also no significant differences in major adverse cardiovascular and cerebrovascular events (11.0% vs. 10.0%; P=1.000), major bleeding (1.0% vs. 3.0%; P=0.616) or BARC 2 bleeding (5.0% vs. 2.0%; P=0.442) between the r-SAK and control groups.

### DISCUSSION

The OPTIMA-5 study demonstrated that in patients presenting within 12 hours of symptoms of STEMI who were expected to receive PCI within 120 minutes of presentation, a single bolus r-SAK administered prior to timely PCI increased patency of the IRA and reduced the infarct size, with no increase in bleeding.

It has been reported that every minute of delay in treatment of patients with STEMI does affect 1-year mortality, not only in primary angioplasty but also in thrombolytic therapy.⁶ The European Society of Cardiology (ESC) guideline recommends that if PCI cannot be performed within 120 minutes of presentation, the thrombolytic therapy should be initiated within 10 minutes of the STEMI diagnosis before initiating PCI, and in patients who are expected to receive PCI within 120 minutes from the FMC, only PCI is recommended for the revascularization treatment. However, the latest meta-analysis showed that in US, Canada, and France, the mean door to balloon times are 66.5 minutes, 78.5 minutes and 72.0 minutes respectively in the pre-pandemic period, and in most of the countries this time is more than 60 minutes. The question is whether we should initiate thrombolysis in the earliest possible time as well for STEMI patients who are expected to receive PCI within 120 minutes, but for more than 60 minutes currently in most countries.

Our study is consistent with previous trials of facilitated PCI that significantly improved TIMI flow of the IRA after thrombolysis. However, our results contradict few studies on infarct size, which has been advocated as an endpoint in reperfusion trials due to its potential prognostic value. Thiele et al found that early administration of reteplase plus abciximab does not lead to a reduction of infarct size compared with abciximab alone in patients with AMI referred for PCI. Kastrati et al reported that In STEMI patients presenting early after symptom onset with relatively long transfer times, a fibrinolytic-based facilitated PCI approach with optimal antiplatelet comedication does not offer a benefit over primary PCI with respect to infarct size and tissue perfusion. As suggested by the investigators, the benefit of a higher infarct artery patency rate upon initial angiography in the facilitated PCI group might have been offset by the delay in mechanical treatment of the infarct-related artery, and the trend toward greater infarct size and microvascular obstruction in the facilitated PCI group might be attributed to paradoxical fibrinolytic induced platelet aggregation and thrombin-induced platelet activation, despite the early and concomitant use of clopidogrel. In our study, however, the concomitant treatment of ticagrelor would be superior to clopidogrel adopted in previous trials to counteract the fibrinolytic induced platelet aggregation and thrombin-induced platelet activation should it present.

Pu et al reported in Early-Myo study that pharmaco-invasive (PhI) PCI with half-dose alteplase and PCI offered more complete epicardial and myocardial reperfusion when compared with PPCI. However, PhI PCI did not significantly reduce the infarct size. In Early-Myo study, patients received angiography or PCI 90 minutes or 3-24 h after fibrinolysis, while in OPTIMA-5 trial patients were transferred to receive PPCI at a mean time of 49 minutes after fibrinolysis, so the un-recanalized coronary arteries after fibrinolysis would be reopened earlier in OPTIMA-5 than that in Early-Myo.¹⁹ This would contribute to the significant reduction of infarct size in r-SAK (plus PPCI) group compared with that in NS (plus PPCI) group in OPTIMA-5 trial.

There are two major clinical trials that have investigated the effect of facilitated PCI versus primary PCI on clinical outcome. The Assessment of the Safety and Efficacy of a New Treatment Strategy with Percutaneous Coronary Intervention (ASSENT-4 PCI) trial found that full-dose tenecteplase with concomitant antithrombotic therapy preceding PCI by 1-3 hours was associated with more major adverse events than PCI alone in STEMI. Besides, significantly more intracranial strokes were reported in patients assigned facilitated rather than standard PCI during hospital stay. In Enhanced Reperfusion to Stop Events (FINESSE) trial, strategies of up-front abciximab alone or in combination with half-dose reteplase were compared with standard PCI in 2452 patients with STEMI. Though significantly more patients in the group that received reteplase plus abciximab than in the group that received abciximab alone or underwent primary PCI had a TIMI flow grade of 3 before PCI was performed (32.8% vs. 14.1% and 12.0%, respectively), neither facilitation of PCI significantly improved the clinical outcomes. Besides, the incidence of major hemorrhage increased in the facilitated PCI groups in FINESSE trial.

Compared with the above trials, the OPTIMA-5 trial differed in the following aspects. First, we mandated the use of ticagrelor instead of clopidogrel in OPTIMA-5 trial. Studies reported that ticagrelor provides more potent platelet inhibition while similar bleeding risk compared with clopidogrel in fibrinolytic-treated STEMI patients undergoing early PCI. Second, OPTIMA-5 trial did not allow use of GPI unless there was evidence of thrombus at CAG. Instead, GPI administration of abciximab was mandated in FINESSE trial. However, as concluded by FINESSE investigators that it seems unlikely that the combination therapy as it was administered in the FINESSE study will be adopted in clinical practice because of the major risk of bleeding associated with this therapy (occurring in more than 25 of 1000 patients treated). Third, half-dose thrombolysis was adopted in OPTIMA-5 study instead of full-dose in ASSENT-4 PCI trial to decrease the bleeding. Fourth, staphylokinase (SAK) is produced by Staphylococcus aureus, which is a third generation, indirect thrombolytic agents. The r-SAK adopted in OPTIMA-5 trial is a recombinant form of SAK, which has been approved by China Food and Drug Administration for treatment of patients with STEMI. Data shows that r-SAK is more potent than urokinase and recombinant streptokinase in fibrinolytic activity, and significantly more fibrin-selective than rt-PA. All the above factors could have contributed to the following results: 1) the TIMI flow grade 3 was 43% in ASSENT-4 PCI trial and 32.8% in FINESSE trial in the thrombolysis groups, while it reached 51% in r-SAK group in OPTIMA-5. 2) the major bleeding was increased in the thrombolysis group in both ASSENT-4 PCI and FINESSE trials, but not in OPTIMA-5. 3) The infarct size was significantly reduced in r-SAK group compared with the control group in OPTIMA-5 trial, which has never been reported in previous trials on facilitated PCI.

Recently, McCartney P J et al performed a T-Time study in which they included patients presenting with STEMI and an ischemic time ≥4 to 6 hours, after successful reperfusion of the IRA, the participants immediately received intracoronary infusion of placebo, alteplase 10 mg, or alteplase 20 mg before stent implantation. Inconsistent with our study results, they found that adjunctive treatment with low-dose intracoronary alteplase during PPCI was associated with increased MVO. However, unlike the T-Time study, the thrombolytic agent was administered intravenously and at a mean time of 49 minutes before PPCI in OPTIMA-5 study. Besides, ticagrelor was mandated in OPTIMA-5, while it was used in less than 6% of the participants in T-Time study. Differences in thrombolytic agent, timing and method of thrombolytic agent administration, and concomitant treatment may have contributed to the differences in the results of the two studies.

OPTIMA-5 trial is the first to investigate the efficacy of a modified or contemporary facilitated PCI with half-dose r-SAK in combination with contemporary antiplatelet agent (ticagrelor) and shortened needle-to-balloon time, and demonstrate that in STEMI patients who are expected to undergo PPCI within 120 minutes of presentation, a single bolus half-dose r-SAK thrombolysis preceded to timely PCI can improve the TIMI flow of IRA before PCI, which is associated with significantly reduced infarct size and a favorable trend of all other minor efficacy endpoints without increasing bleeding.

### Limitations

First, this study was performed during the COVID-19 pandemic, and the patient volume significantly decreased in the 8 centers as a large proportion of the STEMI patients have been treated with thrombolysis at local hospitals. Besides, though this study was started on Oct. 29, 2021 in the Principal Investigation center, other centers started late, with the latest center started in May, 2022. Both of the above issues would have caused potential patient selection bias. Second, a post-hoc approach was adopted to analyze the primary endpoints for patients who underwent PCI within 60 minutes or could not accept CAG due to any reason. Third, investigators were not blinded to treatment allocation. However, images from the coronary artery angiographies were centrally adjudicated by ERC members blinded to the treatment strategy, and the results adjudicated by the ERC were consistent with those assessed by the investigators. Besides, images from the CMR scan were analyzed by an independent core laboratory blinded to treatment allocation. Fourth, although prognostically important, the primary endpoint of TIMI flow grade and the reduction in infarct size are surrogates for clinical events and this study is not powered to demonstrate the impact on patient's clinical outcomes at 1-month or 1-year follow-up. However, this can be a reference for future large-scale clinical trials exploring the impact of the same treatment strategy on clinical outcomes. Fifth, though no significant difference between groups, there were more left anterior descending artery and less circumflex culprits in the control group, which would favor a lower infarct size in the r-SAK group, and it is not possible to exclude differences in patients' characteristics between those who received CMR scan and those who did not as an explanation for differences in infarct size. Sixth, we restricted inclusion to patients aged 18 to 75 years old and it is unclear whether our findings can be extrapolated to older patients.

### CONCLUSIONS

For STEMI patients with symptom onset ≤12 hours expected to undergo PCI within 120 minutes of presentation, a single bolus of half-dose r-SAK improved patency of the IRA and reduced infarct size without increasing major bleeding.

### Example 2: Randomized Study Evaluation of r-SAK Administration (OPTIMA-6)

### Executive Summary:

OPTIMA-6 is a multi-center, randomized, double-blind, placebo-controlled, and superiority trial to evaluate the efficacy of a bolus of half-dose recombinant staphylokinase (r-SAK) vs. placebo prior to timely primary PCI in patients with STEMI. Enrollment began in April 2023 and is expected to enroll 2260 patients at approximately 50 centers. Patients with acute STEMI presenting ≤12 hours of symptom onset and expected to undergo primary PCI within 120 minutes but more than 30 minutes are to be randomized to a bolus of half-dose r-SAK or placebo. All recruited patients will be mandatory to take aspirin and ticagrelor and receive a bolus of loading dose heparin before the thrombolytic therapy. The primary efficacy endpoint is major adverse cardiovascular events (MACE) within 90 days, and the MACE is defined as a composite of all-cause death, reinfarction, unplanned target vessel revascularization, heart failure or cardiogenic shock, and major ventricular arrhythmia. The primary safety endpoints are major bleeding events (BARC 3, 5) within 90 days. OPTIMA-6 will reveal the efficacy and safety of a contemporary facilitated PCI with a bolus of half-dose r-SAK in combination with ticagrelor in patients with STEMI.

### Study Design and Population

OPTIMA-6 is a prospective, multi-center, randomized, double-blind, placebo-controlled, and superiority trial to evaluate the efficacy of a bolus of half-dose r-SAK prior to primary PCI in patients with acute STEMI, presenting ≤12 hours of symptom onset and expected to undergo primary PCI within 120 minutes but more than 30 minutes. This trial has enrolled 791 patients since Apr. 8, 2023 and is planned to enroll 2260 patients at 64 sites. When 60% of the expected patients complete the study, an interim analysis will be carried out by the third-party statisticians to evaluate whether the study can be prematurely terminated. This trial is registered at clinicaltrials.gov (ID number: NCT05410925).

Patients aged 18-75 years and weighing ≥45 kg will be enrolled if they present with STEMI ≤12 hours, with primary PCI expected to be performed ≥30 minutes, and ≤120 minutes. The detailed inclusion and exclusion criteria are listed in Table B1.

TABLE B1
OPTIMA-6 inclusion/exclusion criteria
Inclusion criteria

| 1. | Age 18-75 years, weight ≥45 kg | |
|---|---|---|
| 2. | Diagnosed as STEMI (meeting the following two criteria simultaneously): | |
| | i. | Ischemic chest pain lasts ≥30 minutes |
| | ii. | Electrocardiogram indicates that ST-segment elevation ≥2 mm in 2 or more |
| | | contiguous precordial leads or ≥1 mm in 2 or more peripheral leads |

| | |
|---|---|
| 3. | Time from onset of persistent chest pain to randomization ≤12 hours |
| 4. | Primary PCI expected to be performed ≥30 minutes, and ≤120 minutes |

Exclusion criteria

| | |
|---|---|
| 1. | Cardiogenic shock |
| 2. | Active bleeding or known at high risk of bleeding (including grade III or IV |
| | retinopathy or retinal gastrointestinal or urinary tract hemorrhage within the past |
| | 1 month) |
| 3. | Ischemic stroke or TIA in the past 6 months |
| 4. | History of hemorrhagic stroke |
| 5. | Known intracranial aneurysm |
| 6. | Severe trauma, surgery or head injury within 1 month |
| 7. | Suspected aortic dissection or infective endocarditis |
| 8. | Puncture with difficult hemostasis by compression within 1 month (eg, visceral |
| | biopsy, compartment puncture) |
| 9. | Currently taking anticoagulants |
| 10. | Poorly controlled hypertension (≥180/110 mmHg) |
| 11. | Severe hepatic or renal impairment indicated by the consultation or previous |
| | history (glutamic-pyruvic transaminase or glutamic oxalacetic transaminase >3 |
| | times upper limit of normal value; eGFR <15 ml/min/1.73 m², calculated based |
| | on CKD-EPI) |
| 12. | Known allergy to r-SAK |
| 13. | Pregnancy, lactation, or planning for pregnancy |
| 14. | History of chronic total occlusion, myocardial infarction or CABG |
| 15. | Having taken antiplatelet drugs other than aspirin and ticagrelor, such as |
| | clopidogrel, prasugrel or cilostazol after the symptom onset |
| 16. | Patients with other conditions that made them unsuitable to be recruited at the |
| | discretion of the investigators |

### Treatment Protocol and Follow-Up Procedures

### Treatment Selection

All recruited patients will be treated with a loading dose of aspirin 300 mg and ticagrelor 180 mg, and an intravenous bolus of unfractionated heparin (60 U/kg, and ≤5000 U). Eligible patients will be randomized in a 1:1 ratio to receive either an intravenous bolus of r-SAK 5 mg or matching placebo over 3 minutes. The study drug will be blindly assigned through a central computerized interactive web response system (FIG. 7). The use of platelet glycoprotein IIb/IIIa receptor inhibitor (GPI) or low molecular weight heparin (LMWH) is not recommended unless there is evidence of heavy thrombus burden or no/slow reflow in coronary angiography (CAG). All patients are recommended to receive optimal background medical therapy according to guidelines.

### Coronary Intervention

The protocol requires measurement of the activated clotting time (ACT) after sheath insertion, and dose adjustment of the unfractionated heparin should be performed to achieve a value of ACT between 250 and 300 seconds. For assessment of thrombolysis in myocardial infarction (TIMI) flow grade, CAG is required to be set at 15 frames per second (normal). The duration of angiography at each projection requires ≥5 cardiac cycles (or until contrast agent is fully drained), and the projections are required as follows: 1) RAO 30°, CAU 30°; 2) RAO 30°, CRA 30° if the infarct-related artery (IRA) is the left coronary artery; 1) LAO 40°, CRA 0°; 2) LAO 30°, CRA 30° if the IRA is the right coronary artery. If the TIMI flow of IRA is grade 0-2 in the first CAG, rescue PCI should be performed immediately, while if TIMI flow is grade 3, further revascularization strategy is at the discretion of the attending cardiologists. The following details during PCI are to be recorded in the electronic data capture (EDC) system: date of operation, angiographic results, lesion characteristics, stent type and size, heparin dosage, contrast media dosage, and final results. All CAG images should be digitally stored in digital imaging and communications in medicine (DICOM) format and labelled with the center and patient ID numbers for subsequent offline analyses by independent reviewers blinded to treatment allocation.

### Cardiac Magnetic Resonance

Cardiac magnetic resonance (CMR) scanning (3.0 tesla) will be performed 5 days after randomization in selected centers. The scanning sequences include cine MR, dark-blood T2 weighted imaging, T2 mapping, T1 mapping and late gadolinium enhancement.

Data are required to be stored in DICOM format for subsequent analyses by an independent core laboratory blinded to treatment allocation. The infarct size, microvascular obstruction (MVO), left ventricular ejection fraction (LVEF) and intramuscular hemorrhage (IMH) will be assessed.²⁵

### Follow-Up

All study participants will be scheduled for follow-up at 30±3 days, 90±3 days, 180±7 days, 270±7 days and 360±7 days after enrollment. Follow-up will be conducted by telephone or outpatient visits, as shown in FIG. 8.

### Study Endpoints

The primary efficacy endpoint is defined as a major adverse cardiovascular event (MACE) within 90 days, including all-cause death, reinfarction, unplanned target revascularization, heart failure or cardiogenic shock, and major ventricular arrhythmias. The secondary efficacy endpoints are listed in Table B2, mainly including each clinical outcome of MACE, N-terminal pro-B-type natriuretic peptide (NT-proBNP), indexes of echocardiogram and CMR, and TIMI flow grade before and after PCI. The primary safety endpoints are major bleeding events (BARC 3, 5) within 90 days, and the minor bleeding events (BARC 2) should also be recorded.

The event review committee (ERC) that is blinded to treatment allocation, independent of the study team, will review all primary endpoints. All researchers need to receive training in event review as a prerequisite for trial qualification. This training includes instruction on the accurate assessment and documentation of clinical endpoints, adverse and severe adverse events. All endpoint definitions adopted in OPTIMA-6 are provided below.

### 1. Cardiovascular (CV) Death¹

Including sudden cardiac death, death due to acute myocardial infarction (AMI), death due to heart failure or cardiogenic shock, death due to stroke and cerebrovascular events, death due to other cardiovascular causes.

### 1) Sudden Cardiac Death

a) Witnessed and instantaneous without new or worsening symptoms;
b) Witnessed within 60 minutes of the onset of new or worsening cardiac symptoms;
c) Witnessed and attributed to an identified arrhythmia (eg, captured on electrocardiogram (ECG) recording or witnessed on a monitor by either a medic or paramedic);
d) Subjects unsuccessfully resuscitated from cardiac arrest or successfully resuscitated from cardiac arrest but who die within 24 hours without identification of a non-cardiac Etiology;
e) Unwitnessed death and there is no conclusive evidence of another, non-CV, cause of death (ie, presumed CV death).

### 2) Sudden Death Due to AMI

Sudden death occurring up to 14 days after a documented AMI (verified either by the universal definition of AMI or by autopsy findings showing recent myocardial infarction or recent coronary thrombus) and where there is no conclusive evidence of another cause of death. If death occurs before biochemical confirmation of myocardial necrosis can be obtained, adjudication should be based on clinical presentation and ECG evidence.

### 3) Death Due to Heart Failure or Cardiogenic Shock

New or worsening signs and/or symptoms of heart failure include any of the following:
a) New or increasing symptoms and/or signs of heart failure requiring the initiation of, or an increase in, treatment directed at heart failure or occurring in a patient already receiving maximal therapy for heart failure;
b) Heart failure symptoms or signs requiring continuous intravenous therapy or oxygen administration;
c) Confinement to bed predominantly due to heart failure symptoms;
d) Pulmonary edema sufficient to cause tachypnea and distress not occurring in the context of an acute myocardial infarction or as the consequence of an arrhythmia occurring in the absence of worsening heart failure;
e) Cardiogenic shock not occurring in the context of an AMI or as the consequence of an arrhythmia occurring in the absence of worsening heart failure.
   Cardiogenic shock is defined as systolic pressure (SBP)<90 mmHg for more than 1 hour, not responsive to fluid resuscitation and/or heart rate correction, and felt to be secondary to cardiac dysfunction and associated with at least one of the following signs of hypoperfusion:
      Cool, clammy skin
      Oliguria (urine output <30 mL/h)
      Altered sensorium
      Cardiac index <2.2 L/min/m²
   Cardiogenic shock can also be defined in the presence of SBP ≥90 mmHg or for a time period <1 hour if the blood pressure measurement or the time period is influenced by the presence of positive inotropic or vasopressor agents alone and/or with mechanical support <1 hour.

### 4) Death Due to Stroke, Cerebrovascular Events

Death occurring up to 30 days after a stroke that is either due to the stroke or caused by complication of the stroke.

### 5) Death Due to Other CV Causes

Death must be due to a fully documented CV cause not included in the above categories (eg, dysrhythmia, pulmonary embolism, or CV intervention). Death due to a myocardial infarction that occurs as a direct consequence of a CV investigation/procedure/operation will be classified as death due to other CV cause.

### 2. Non-CV Death¹

Non-CV death is defined as any death not covered by cardiac death or vascular death. Examples of non-CV death are: pulmonary causes, renal causes, gastrointestinal causes, infection (including sepsis), non-infectious (eg, systemic inflammatory response syndrome), malignancy (ie, new malignancy, worsening of prior malignancy), hemorrhage (not intracranial), accidental/trauma, suicide, non-CV organ failure (eg, hepatic failure) or non-CV surgery.

### 3. Reinfarction

1) In the first 18 hours after randomization reinfarction is defined as:

Recurrent signs and symptoms of ischemia at rest, accompanied by new or recurrent ST-segment elevations of ≥0.1 mV in at least two contiguous leads lasting ≥30 minutes.
2) After 18 hours reinfarction is defined as follows:
   a) New Q waves (by Minnesota Code Criteria) in two or more leads and/or enzyme/biochemical evidence of reinfarction: re-elevation of creatine kinase myocardial band (CK-MB) or troponin to above the upper limit of normal and increased by ≥50% over the previous value;
   b) If CK-MB or troponin is not available, the total creatine kinase (CK) will be evaluated:
      The total CK must either be re-elevated to ≥2 times the upper limit of normal and increased by ≥25% or be re-elevated to ≥200 U/ml over the previous value;
      If re-elevated to <2 times the upper limit of normal, the total CK must exceed the upper limit of normal by ≥50% and exceed the previous value by two-fold or be re-elevated to ≥200 U/ml.
3) Reinfarction after percutaneous coronary intervention (PCI) is defined as:

CK-MB (or CK, if CK-MB is not available) >3 times the upper limit of normal and >50% greater than the previous value and/or new Q waves (Minnesota Code) in two or more contiguous leads.
4) Reinfarction after coronary artery bypass grafting (CABG) surgery is defined as:

CK-MB (or CK, if CK-MB is not available) >5 times the upper limit of normal and >50% greater than the previous value and/or new Q waves (Minnesota Code) in two or more contiguous leads.

### 4. Target Vessel Revascularization (TVR)

TVR is defined as any repeat percutaneous intervention or surgical bypass of any segment of the target vessel. The target vessel is defined as the entire major coronary vessel proximal and distal to the target lesion, which includes upstream and downstream branches and the target lesion itself.

### 5. Heart Failure and Cardiogenic Shock

1) Heart failure (consists of at least one of the following conditions requiring treatment with diuretics)
   a) Pulmonary oedema/congestion on chest X-ray without suspicion of a non-cardiac cause;
   b) Rales >⅓ up from the lung base (Killip class 2 or higher);
   c) Pulmonary capillary wedge pressure (PCWP) >25 mmHg;
   d) Dyspnea with pO₂<80 mmHg or O₂ sat <90% (no supplemental O₂) in the absence of known lung disease.
2) Cardiogenic shock (defined as one of the following)
   a) Systolic blood pressure <90 mmHg for at least 30 minutes (or the need for supportive measures to maintain a systolic blood pressure of >90 mmHg) in the presence of a heart rate of >60 beats/min in association with signs of end organ hypoperfusion (cold extremities, low urinary output <30 mL/h and/or mental confusion);
b) A cardiac index <2.2 L/min/m² in the presence of a PCWP of >15 mmHg.

### 6. Major Ventricular Arrhythmias⁵

Ventricular arrhythmias >6 hours after randomization requiring electrical cardioversion/defibrillation.

### 7. Cardiac Mechanical Complications⁶

Defined as ventricular septal rupture, papillary muscle rupture, free-wall rupture or ventricular aneurysm diagnosed by echocardiography after AMI.
1) Ventricular septal rupture: Within 7 days after AMI; Echocardiography shows shunt flow across ventricular septum and simple apical defect or extensive irregular inferobasal defect;
2) Papillary muscle rupture: Within 7 days after AMI; Echocardiography shows ruptured papillary muscle, prolapse/flail leaflets, or severe mitral regurgitation;
3) Free-wall rupture: Within 7 days after AMI; Echocardiography shows pericardial effusion/tamponade or pericardial clots;
4) Ventricular aneurysm: After AMI; Echocardiography shows a full-thickness scar that is characterized by a localized, convex protrusion during both phases of the cardiac cycle, and complete loss of contractility as shown by akinesia or paradoxical motion during systole.⁷

### 8. Ischemic Stroke

Defined as any new neurologic deficit lasting >24 hours and presence of ischemic injury confirmed by computed tomography (CT) or magnetic resonance imaging (MRI).

### 9. Bleeding Academic Research Consortium (BARC) Definition for Bleeding

BARC 0: no evidence of bleeding;
BARC 1: bleeding that is not actionable and does not cause the patient to seek unscheduled performance of studies, hospitalization, or treatment by a healthcare professional; may include episodes leading to self-discontinuation of medical therapy by the patient without consulting a healthcare professional;
BARC 2: any overt, actionable sign of hemorrhage (eg, more bleeding than would be expected for a clinical circumstance, including bleeding found by imaging alone) that does not fit the criteria for type 3, 4, or 5 but does meet at least one of the following criteria: (1) requiring nonsurgical, medical intervention by a healthcare professional, (2) leading to hospitalization or increased level of care, or (3) prompting evaluation;
BARC 3:
BARC 3a: significant bleeding with Hb decreased 30-50 g/L, significant bleeding requiring blood transfusion; Any transfusion with overt bleeding;
BARC 3b: bleeding and Hb decrease ≥50 g/L; cardiac tamponade; bleeding that requires surgical treatment or control (excluding teeth, nose, skin and hemorrhoids); bleeding requiring intravenous vasoactive drugs;
BARC 3c: intracranial hemorrhage (excluding trace cerebral hemorrhage and hemorrhagic transformation after cerebral infarction, including intraspinal hemorrhage); Subtypes confirmed by autopsy, imaging examination and lumbar puncture; Intraocular bleed compromising vision;
BARC 4: CABG-related bleeding
(1) Perioperative intracranial hemorrhage within 48 hours;
(2) Reoperation to control bleeding after sternotomy;
(3) Transfusion of ≥5 units of whole blood or concentrated red blood cells within 48 hours;
(4) ≥2 L drainage through chest catheter within 24 hours;
BARC 5: fatal bleeding
BARC 5a: clinically suspicious fatal bleeding not confirmed by autopsy or imaging;
BARC 5b: definite fatal hemorrhage confirmed by autopsy or imaging.
Major bleeding: BARC ≥3;
Minor bleeding: BARC 1 and 2.

### Statistical Considerations

All eligible patients will be assigned randomly in a 1:1 ratio to receive r-SAK or placebo treatment, respectively. Efficacy analyses will be conducted on all randomized patients according to intention-to-treat principle. Safety analyses will be conducted on patients with at least one safety assessment after randomization. A two-sided χ² test or exact Fisher test will be used to assess the difference in the primary endpoint between r-SAK and placebo groups, which will be presented in the Kaplan-Meier curves and the result of log-rank test as well. The primary endpoint will also be evaluated in a number of subgroup analyses according to the baseline factors, including time from symptom onset to thrombolysis, time from thrombolysis to CAG, IRA, infarct location etc. The secondary endpoints will be tested hierarchically in the order listed in Table B2. Measurement data in the secondary endpoints will be tested using t test, and enumeration data will be tested using a two-sided χ² test or exact Fisher test.

TABLE B2
OPTIMA-6 trial endpoints

Primary efficacy endpoint
MACE defined as a composite of all-cause death, reinfarction, unplanned target
vessel revascularization, heart failure or cardiogenic shock, major ventricular
arrhythmia (within 90 days)
Secondary efficacy endpoints
Each of the following cardiac and cerebrovascular events within 90 days: all-
cause death, cardiovascular death, reinfarction, ischemic stroke, unplanned
target vessel revascularization, heart failure or cardiogenic shock, major
ventricular arrhythmia, cardiogenic death, other cardiogenic rehospitalization,
mechanic complications including ventricular septal rupture, papillary muscle
rupture, cardiac rupture, and ventricular aneurysm
NT-proBNP (1 day before discharge or day 7, day 90 ± 3)
Infarct size, LVEF, MVO and IMH, assessed by CMR (day 5)
LVEF assessed by echocardiogram using Simpson's method (day 90 ± 3)
The percentage of TIMI flow grade 2 and 3 prior to PCI
The percentage of TIMI flow grade 3 prior to PCI
The percentage of TIMI flow grade 2 and 3 after PCI
The percentage of TIMI flow grade 3 after PCI
MACE defined as a composite of all-cause death, reinfarction, unplanned target
vessel revascularization, heart failure or cardiogenic shock, major ventricular
arrhythmia (within 360 days)
Each of the following cardiac and cerebrovascular events: all-cause death,
cardiovascular death, reinfarction, ischemic stroke, unplanned target vessel
revascularization, heart failure or cardiogenic shock, major ventricular
arrhythmia, cardiogenic death, other cardiogenic rehospitalization, mechanic
complications including ventricular septal rupture, papillary muscle rupture,
cardiac rupture, and ventricular aneurysm (within 360 days)
NT-proBNP (day 360 ± 7)
LVEF assessed by echocardiogram using Simpson's method (day 360 ± 7)
Safety endpoints
Major bleeding events during hospitalization or within 7 days (BARC 3, 5)
Minor bleeding events during hospitalization or within 7 days (BARC 2)
Major bleeding events within 90 days (BARC 3, 5)
Minor bleeding events within 90 days (BARC 2)

An interim analysis will be performed after 60% of the expected patients complete the study to allow for the study to be terminated prematurely owing to either efficacy or futility using a pre-specified boundary (two-sided nomial α was 0.001 and 0.050, respectively).²⁶ Incorporating these elements and assuming the incidence of the primary endpoint of 16% in the placebo group,^{17,26} a maximum of 2214 patients are required to detect a 28% relative or 4.48% absolute risk reduction of MACE with r-SAK, with 85% power at a two-sided significance level of 5%. Assuming an additional drop-out rate of 2%, it is determined to enroll 2260 patients maximumly for OPTIMA-6, and the trial will be terminated prematurely if extremely significant reduction of MACE is detected at the interim analysis.

### Ethical Considerations

The OPTIMA-6 trial complied with the Declaration of Helsinki, the International Council for Harmonization Guidelines for Good Clinical Practice. In accordance with national and international regulations, the ethics committee or relevant institutional review boards must review and approve the full protocol before initiating the trial at designated sites. This study has been approved by the Research Ethics Board of the First Affiliated Hospital of Nanjing Medical University (2022-SR-567). Written informed consent must be obtained from each participant before enrollment.

### Discussion

The OPTIMA-6 trial is a prospective, multi-center, randomized, double-blind, placebo-controlled trial designed to answer the hypothesis that a bolus of half-dose r-SAK compared with placebo prior to primary PCI could improve clinical outcomes in patients with acute STEMI when primary PCI is expected to be performed within 120 minutes but more than 30 minutes. The primary endpoint of the study is the 90-day incidence of MACE defined as a composite of all-cause death, reinfarction, unplanned target vessel revascularization, heart failure or cardiogenic shock, major ventricular arrhythmia.

The core of STEMI treatment is to achieve early reperfusion of IRA and minimize the total time of myocardial ischemia, which is the consideration of original intention of implementing facilited PCI, especially in scenarios where timely access to primary PCI is not available.

A few studies have investigated the efficacy of facilitated PCI with full- or reduced-dose thrombolytic therapy in STEMI, but no significant benefit of clinical outcomes was found compared with primary PCI alone. The ASSENT-4 trial showed that full-dose tenecteplase administered 1 to 3 hours prior to PCI was associated with more major adverse events than PCI alone. By comparison, the FINESSE trial investigated the efficacy of facilitated PCI with half-dose reteplase plus abciximab or abciximab compared with primary PCI alone, and still no benefit of MACE was found, though the TIMI flow grade prior to PCI significantly improved in the reteplase facilitated PCI group. A meta-analysis of small studies of facilitated PCI, which together showed that facilitated PCI improved pre-PCI TIMI flow grade but did not affect clinical outcomes, in line with FINESSE and ASSENT-4.²¹

Why earlier reperfusion strategy with thrombolysis did not bring clinical benefits? First, thrombolytic agents used in previous studies, such as tenecteplase and reteplase, are not potent enough to achieve satisfactory TIMI flow prior to PCI, with 43% of TIMI flow grade 3 in ASSENT-4 trial and 32.8% in FINESSE trial, the study results would be different if higher reperfusion rate were achieved. Second, the absence of optimum antithrombotic co-therapy may predispose to imbalance of thrombosis and bleeding. Lack of up-front loading dose of clopidogrel resulted in an excess of early thrombotic complications in the ASSENT-4 trial, while the combined use with GPI increased the incidence of major hemorrhage in the FINESSE trial.¹⁹ Third, the time interval between thrombolysis and PCI was too long, so that in most of the patients with non-reopened coronary artery, the infarct size would be extended and more cardiac myocyte would not be salvageable by PCI. The median time from thrombolysis to PCI was 104 minutes in ASSENT-4 trial and 90 minutes in FINESSE trial, both have showed significant treatment delays.

In OPTIM-6 trial, however, we have modified the conventional facilitated PCI strategies in the following aspects, which we call contemporary facilitated PCI. First, we will adopt another third-generation thrombolytic agent of r-SAK characterized by high fibrinolytic activity and fibrin selectivity, and a half-dose r-SAK will be administered to reduce the risk of bleeding. It has been demonstrated that a single bolus of r-SAK could recanalize the IRA within 30 minutes of thrombolysis with superior efficacy over urokinase and recombinant streptokinase. Second, the new P2Y₁₂ inhibitor, ticagrelor, which is more potent than clopidogrel, would be mandatorily administered in OPTIMA-6 to suppress transient platelet activation immediately following thrombolysis and thereby prevent thrombotic re-occlusion.^{31,32} In accordance with the current guidelines, the use of GPI is not recommended in OPTIMA-6 unless there is evidence of heavy thrombus burden or no/slow reflow in CAG, by which we aim to lower the risk of bleeding. Third, as every minute of delay in PCI is associated with larger infarct size and worse clinical outcomes, and the risk of 1-year mortality would be increased by 7.5% for each 30-minute delay. In OPTIMA-6 trial, PCI is required to be performed as early as possible within 120 minutes after thrombolysis in order to salvage the ischemic myocardium once the IRA is not recannalized, though the thrombolytic treatment is to be initiated in patients who are expected to undergo primary PCI 30 minutes later to allow the r-SAK to make effects to achieve an earliest possible reperfusion.

The comparison of the OPTIMA-6 study with the ASSENT-4 and FINESSE studies in terms of study design and the incidence of MACE is summarized in Table B3. The data demonstrate that the overall all-cause mortality and major bleeding rates in the OPTIMA-6 study are lower than those observed in the other two studies, highlighting the superior efficacy and safety of the contemporary facilitated PCI strategy.

TABLE B3
Comparison of trial designs and MACE rates for facilitated PCI

| | | | Thrombolysis | | | | |
|---|---|---|---|---|---|---|---|
| | | | to PCI | | Concomitant | | |
| | | No. of | Interval | Thrombo- | antiplatelet | Concomitant | MACE |
| Trial | Treatment | subjects | (min) | lytics | agents | GPI | incidence |
| ASSENT-4 | Facilitated | 829 | 104 | Tenecteplase | Aspirin + | At the | 18.6% |
| | PCI | | | | clopidogrel | discretion | |
| | Primary PCI | 838 | | | | of | 13.4% |
| | | | | | | investigators | |
| FINESSE | Facilitated | 828 | 90 | Reteplase | Aspirin | Abciximab | 9.8% |
| | PCI | | | | | | |
| | Primary PCI | 806 | | | | | 10.7% |
| OPTIMA-6 | Facilitated | 2260 | 30-120 | r-SAK | Aspirin + | Not | 13.6% |
| | PCI | (785 have | | | ticagralor | recommended | |
| | Primary PCI | been | | | | to use | |
| | | enrolled) | | | | | |

| | | | | | | Major | |
|---|---|---|---|---|---|---|---|
| | | All-cause | Reinfarc- | Unplanned | HF | ventricular | Major |
| Trial | Treatment | death | tion | IVR | or CS | arrhythmia | bleeding |
| ASSENT-4 | Facilitated | 55 (6.6%) | 49 (5.9%) | 53 (6.4%) | 148 (17.9%) | NA | 46 (5.6%) |
| | PCI | | | | | | |
| | Primary PCI | 41 (4.9%) | 30 (3.6%) | 28 (3.3%) | 115 (13.7%) | NA | 37 (4.4%) |
| FINESSE | Facilitated | 43 (5.2%) | 17 (2.1%) | 12 (1.4%) | 60 (7.2%) | 5 (0.6%) | 40 (4.8%) |
| | PCI | | | | | | |
| | Primary PCI | 36 (4.5%) | 15 (1.9%) | 15 (1.9%) | 73 (9.0%) | 3 (0.4%) | 21 (2.6%) |
| OPTIMA-6 | Facilitated | 22 (2.8%) | 6 (0.8%) | 13 (1.7%) | 88 (11.2%) | 5 (0.6%) | 15 (1.9%) |
| | PCI | | | | | | |
| | Primary PCI | | | | | | |

As a matter of fact, we have performed a pilot study of OPTIMA-5 with the same treatment strategy as that in OPTIMA-6, and found that in STEMI patients who were expected to receive PCI within 120 minutes, a single bolus of half-dose r-SAK in combination with ticagrelor prior to primary PCI significantly improved the rate of TIMI flow grade 2 to 3 (69.0% vs. 29.0%, P<0.001) and reduced the infarct size (21.91±10.84% vs. 26.85±12.37%, P=0.016) without increasing major bleeding (0.0% vs. 3.0%, P=0.246) compared with PCI alone (in press). On this basis, the OPTIMA-6 trial mainly aimed to investigate the efficacy of the contemporary facilitated PCI on the clinical outcomes in STEMI patients during 90-day follow-up.

In view of the favorable results of OPTIMA-5, a group sequential design is adopted in the OPTIMA-6 trial. When the proportion of patients who complete the study reaches 60%, an interim analysis will be carried out. The double-blind study design, objective endpoints with clearly defined criteria, strict data management based on EDC system, advanced training of investigators, and the supervision of DSMC will ensure the data quality and thus meaningful results.

### Conclusions

The OPTIMA-6 trial will reveal the efficacy and safety of a contemporary facilitated PCI with a single bolus of half-dose r-SAK in combination with ticagrelor in patients with STEMI who are expected to undergo primary PCI within 120 minutes but more than 30 minutes. All patents, patent publications, and other publications herein are incorporated herein by reference.

### REFERENCES

Armstrong P W, Gershlick A H, Goldstein P, Wilcox R, Danays T, Lambert Y, Sulimov V, Rosell Ortiz F, Ostojic M, Welsh R C, et al. Fibrinolysis or primary PCI in ST-segment elevation myocardial infarction. N Engl J Med. 2013; 368:1379-1387.
Assessment of the S, Efficacy of a New Treatment Strategy with Percutaneous Coronary Intervention i. Primary versus tenecteplase-facilitated percutaneous coronary intervention in patients with ST-segment elevation acute myocardial infarction (ASSENT-4 PCI): randomised trial. Lancet. 2006; 367:569-578.
Bahit M C, Kochar A, Granger C B. Post-Myocardial Infarction Heart Failure. JACC Heart Fail. 2018; 6:179-186.
Berwanger O, Nicolau J C, Carvalho A C, Jiang L, Goodman S G, Nicholls S J, Parkhomenko A, Averkov O, Tajer C, Malaga G, et al. Ticagrelor vs Clopidogrel After Fibrinolytic Therapy in Patients With ST-Elevation Myocardial Infarction: A Randomized Clinical Trial. JAMA Cardiol. 2018; 3:391-399.
Bhatt D L, Lopes R D, Harrington R A. Diagnosis and Treatment of Acute Coronary Syndromes: A Review. JAMA 2022; 327(7):662-675.
Brener S J, Mehran R, Brodie B R, Guagliumi G, Witzenbichler B, Cristea E, Xu K, Lansky A J, Stone G W. Predictors and implications of coronary infarct artery patency at initial angiography in patients with acute myocardial infarction (from the CADILLAC and HORIZONS-AMI Trials). Am J Cardiol. 2011; 108:918-923.
Bueno H, Betriu A, Heras M, Alonso J J, Cequier A, Garcia E J, et al. Primary angioplasty vs. fibrinolysis in very old patients with acute myocardial infarction: TRIANA (TRatamiento del Infarto Agudo de miocardio eN Ancianos) randomized trial and pooled analysis with previous studies. Eur Heart J 2011; 32(1):51-60.
Carrick D, Haig C, Ahmed N, Carberry J, Yue May V T, McEntegart M, et al. Comparative Prognostic Utility of Indexes of Microvascular Function Alone or in Combination in Patients With an Acute ST-Segment-Elevation Myocardial Infarction. Circulation 2016; 134(23):1833-1847.
Chesebro J H, Knatterud G, Roberts R, Borer J, Cohen L S, Dalen J, Dodge H T, Francis C K, Hillis D, Ludbrook P, et al. Thrombolysis in Myocardial Infarction (TIMI) Trial, Phase I: A comparison between intravenous tissue plasminogen activator and intravenous streptokinase. Clinical findings through hospital discharge. Circulation. 1987; 76:142-154.
Crisan S, Petris A O, Petrescu L, Luca C T. Current Perspectives in Facilitated Angioplasty. Am J Ther 2019; 26(2):e208-e212.
De Luca G, Suryapranata H, Ottervanger J P, Antman E M. Time delay to treatment and mortality in primary angioplasty for acute myocardial infarction: every minute of delay counts. Circulation. 2004; 109:1223-1225.
Dehghani P, Lavoie A, Lavi S, Crawford J J, Harenberg S, Zimmermann R H, Booker J, Kelly S, Cantor W J, Mehta S R, et al. Effects of ticagrelor versus clopidogrel on platelet function in fibrinolytic-treated STEMI patients undergoing early PCI. Am heart J. 2017; 192:105-112.
Ellis S G, Tendera M, de Belder M A, van Boven A J, Widimsky P, Janssens L, et al. Facilitated PCI in patients with ST-elevation myocardial infarction. N Engl J Med 2008; 358(21):2205-17.
Gibbons R J, Miller T D, Christian T F. Infarct size measured by single photon emission computed tomographic imaging with (99m)Tc-sestamibi: A measure of the efficacy of therapy in acute myocardial infarction. Circulation. 2000; 101:101-108.
Group T S. The Thrombolysis in Myocardial Infarction (TIMI) trial. Phase I findings. N Engl J Med 1985; 312(14):932-6.
Heusch G, Libby P, Gersh B, Yellon D, Bohm M, Lopaschuk G, Opie L. Cardiovascular remodelling in coronary artery disease and heart failure. Lancet. 2014; 383:1933-1943.
Ibanez B, James S, Agewall S, Antunes M J, Bucciarelli-Ducci C, Bueno H, Caforio A L P, Crea F, Goudevenos J A, Halvorsen S, et al. 2017 ESC Guidelines for the management of acute myocardial infarction in patients presenting with ST-segment elevation: The Task Force for the management of acute myocardial infarction in patients presenting with ST-segment elevation of the European Society of Cardiology (ESC). Eur Heart J. 2018; 39:119-177.
Ibanez B, James S, Agewall S, Antunes M J, Bucciarelli-Ducci C, Bueno H, et al. 2017 ESC Guidelines for the management of acute myocardial infarction in patients presenting with ST-segment elevation. Rev Esp Cardiol (Engl Ed) 2017; 70(12):1082.
Investigators A M. Facilitated percutaneous coronary intervention for acute ST-segment elevation myocardial infarction: results from the prematurely terminated ADdressing the Value of facilitated ANgioplasty after Combination therapy or Eptifibatide monotherapy in acute Myocardial Infarction (ADVANCE MI) trial. Am Heart J 2005; 150(1):116-22.
Kamarullah W, Sabrina A P, Rocky M A, Gozali D R. Investigating the implications of COVID-19 outbreak on systems of care and outcomes of STEMI patients: A systematic review and meta-analysis. Indian Heart J. 2021; 73:404-412.
Kastrati A, Mehilli J, Schlotterbeck K, Dotzer F, Dirschinger J, Schmitt C, Nekolla S G, Seyfarth M, Martinoff S, Markwardt C, et al. Early administration of reteplase plus abciximab vs abciximab alone in patients with acute myocardial infarction referred for percutaneous coronary intervention: a randomized controlled trial. JAMA. 2004; 291:947-954.
Keeley E C, Boura J A, Grines C L. Comparison of primary and facilitated percutaneous coronary interventions for ST-elevation myocardial infarction: quantitative review of randomised trials. Lancet 2006; 367(9510):579-88.
Keeley E C, Boura J A, Grines C L. Primary angioplasty versus intravenous thrombolytic therapy for acute myocardial infarction: a quantitative review of 23 randomised trials. Lancet 2003; 361(9351):13-20.
Kohli P, Wallentin L, Reyes E, Horrow J, Husted S, Angiolillo D J, et al. Reduction in first and recurrent cardiovascular events with ticagrelor compared with clopidogrel in the PLATO Study. Circulation 2013; 127(6):673-80.
Lawton J S, Tamis-Holland J E, Bangalore S, Bates E R, Beckie T M, Bischoff J M, et al. 2021 ACC/AHA/SCAI Guideline for Coronary Artery Revascularization: Executive Summary: A Report of the American College of Cardiology/American Heart Association Joint Committee on Clinical Practice Guidelines. Circulation 2022; 145(3):e4-e17.
Le May M R, Wells G A, Labinaz M, Davies R F, Turek M, Leddy D, et al. Combined angioplasty and pharmacological intervention versus thrombolysis alone in acute myocardial infarction (CAPITAL AMI study). J Am Coll Cardiol 2005; 46(3):417-24.
Li C J, Huang J, Yang Z J, Cao K J. Thrombolytic efficacy of native recombinant staphylokinase on femoral artery thrombus of rabbits. Acta Pharmacol Sin 2007; 28(1):58-65.
Li J, Li X, Wang Q, Hu S, Wang Y, Masoudi F A, Spertus J A, Krumholz H M, Jiang L. ST-segment elevation myocardial infarction in China from 2001 to 2011 (the China PEACE-Retrospective Acute Myocardial Infarction Study): a retrospective analysis of hospital data. Lancet. 2015; 385:441-451.
McCartney P J, Maznyczka A M, Eteiba H, McEntegart M, Oldroyd K G, Greenwood J P, Maredia N, Schmitt M, McCann G P, Fairbairn T, et al. Low-Dose Alteplase During Primary Percutaneous Coronary Intervention According to Ischemic Time. J Am Coll Cardiol. 2020; 75:1406-1421.
Mehran R, Rao S V, Bhatt D L, Gibson C M, Caixeta A, Eikelboom J, Kaul S, Wiviott S D, Menon V, Nikolsky E, et al. Standardized bleeding definitions for cardiovascular clinical trials: a consensus report from the Bleeding Academic Research Consortium. Circulation. 2011; 123:2736-2747.
Nedaeinia R, Faraji H, Javanmard S H, Ferns G A, Ghayour-Mobarhan M, Goli M, et al. Bacterial staphylokinase as a promising third-generation drug in the treatment for vascular occlusion. Mol Biol Rep 2020; 47(1):819-841.
O'Neill W W, Weintraub R, Grines C L, Meany T B, Brodie B R, Friedman H Z, et al. A prospective, placebo-controlled, randomized trial of intravenous streptokinase and angioplasty versus lone angioplasty therapy of acute myocardial infarction. Circulation 1992; 86(6):1710-7.
Peters S, Truemmel M, Koehler B. Facilitated PCI by combination fibrinolysis or upstream tirofiban in acute ST-segment elevation myocardial infarction: results of the Alteplase and Tirofiban in Acute Myocardial Infarction (ATAMI) trial. Int J Cardiol. 2008; 130:235-240.
Pu J, Ding S, Ge H, Han Y, Guo J, Lin R, Su X, Zhang H, Chen L, He B, et al. Efficacy and Safety of a Pharmaco-Invasive Strategy With Half-Dose Alteplase Versus Primary Angioplasty in ST-Segment-Elevation Myocardial Infarction: EARLY-MYO Trial (Early Routine Catheterization After Alteplase Fibrinolysis Versus Primary PCI in Acute ST-Segment-Elevation Myocardial Infarction). Circulation. 2017; 136:1462-1473.
Ren K, Gong H, Huang J, Liu Y, Dong Q, He K, et al. Thrombolytic and anticoagulant effects of a recombinant staphylokinase-hirudin fusion protein. Thromb Res 2021; 208:26-34.
Ross A M, Coyne K S, Reiner J S, Greenhouse S W, Fink C, Frey A, et al. A randomized trial comparing primary angioplasty with a strategy of short-acting thrombolysis and immediate planned rescue angioplasty in acute myocardial infarction: the PACT trial. PACT investigators. Plasminogen-activator Angioplasty Compatibility Trial. J Am Coll Cardiol 1999; 34(7):1954-62.
Schulz K F, Grimes D A. Multiplicity in randomised trials II: subgroup and interim analyses. Lancet 2005; 365(9471):1657-61.
Stone G W, Cox D, Garcia E, Brodie B R, Morice M C, Griffin J, Mattos L, Lansky A J, O'Neill W W, Grines C L. Normal flow (TIMI-3) before mechanical reperfusion therapy is an independent determinant of survival in acute myocardial infarction: analysis from the primary angioplasty in myocardial infarction trials. Circulation. 2001; 104:636-641.
Thiele H, Eitel I, Meinberg C, Desch S, Leuschner A, Pfeiffer D, et al. Randomized comparison of pre-hospital-initiated facilitated percutaneous coronary intervention versus primary percutaneous coronary intervention in acute myocardial infarction very early after symptom onset: the LIPSIA-STEMI trial (Leipzig immediate prehospital facilitated angioplasty in ST-segment myocardial infarction). JACC Cardiovasc Interv 2011; 4(6):605-14.
Thiele H, Engelmann L, Elsner K, Kappl M J, Storch W H, Rahimi K, et al. Comparison of pre-hospital combination-fibrinolysis plus conventional care with pre-hospital combination-fibrinolysis plus facilitated percutaneous coronary intervention in acute myocardial infarction. Eur Heart J 2005; 26(19):1956-63.
Thrane P G, Kristensen S D, Olesen K K W, Mortensen L S, Botker H E, Thuesen L, et al. 16-year follow-up of the Danish Acute Myocardial Infarction 2 (DANAMI-2) trial: primary percutaneous coronary intervention vs. fibrinolysis in ST-segment elevation myocardial infarction. Eur Heart J 2020; 41(7):847-854.
Thygesen K, Alpert J S, Jaffe A S, Chaitman B R, Bax J J, Morrow D A, White H D, Executive Group on behalf of the Joint European Society of Cardiology/American College of Cardiology/American Heart Association/World Heart Federation Task Force for the Universal Definition of Myocardial I. Fourth Universal Definition of Myocardial Infarction (2018). Glob Heart. 2018; 13:305-338.
Townsend N, Wilson L, Bhatnagar P, Wickramasinghe K, Rayner M, Nichols M. Cardiovascular disease in Europe: epidemiological update 2016. European heart journal. 2016; 37:3232-3245.
Tsao C W, Aday A W, Almarzooq Z I, Alonso A, Beaton A Z, Bittencourt M S, et al. Heart Disease and Stroke Statistics-2022 Update: A Report From the American Heart Association. Circulation 2022; 145(8):el53-e639.
Tsao C W, Aday A W, Almarzooq Z I, Anderson C A M, Arora P, Avery C L, Baker-Smith CM, Beaton A Z, Boehme A K, Buxton A E, et al; on behalf of the American Heart Association Council on Epidemiology and Prevention Statistics Committee and Stroke Statistics Subcommittee. Heart Disease and Stroke Statistics-2023 Update: A Report From the American Heart Association. Circulation. 2023; 147:e93-e621.
Vakili B, Nezafat N, Negahdaripour M, Yari M, Zare B, Ghasemi Y. Staphylokinase Enzyme: An Overview of Structure, Function and Engineered Forms. Curr Pharm Biotechnol 2017; 18(13):1026-1037.
Vanderschueren S, Barrios L, Kerdsinchai P, Van den Heuvel P, Hermans L, Vrolix M, De Man F, Benit E, Muyldermans L, Collen D, et al. A randomized trial of recombinant staphylokinase versus alteplase for coronary artery patency in acute myocardial infarction. The STAR Trial Group. Circulation. 1995; 92:2044-2049.
Vermeer F, Oude Ophuis A J, vd Berg E J, Brunninkhuis L G, Werter C J, Boehmer A G, et al. Prospective randomised comparison between thrombolysis, rescue PTCA, and primary PTCA in patients with extensive myocardial infarction admitted to a hospital without PTCA facilities: a safety and feasibility study. Heart 1999; 82(4):426-31.
Wallentin L, Becker R C, Budaj A, Cannon C P, Emanuelsson H, Held C, Horrow J, Husted S, James S, Katus H, et al. Ticagrelor versus clopidogrel in patients with acute coronary syndromes. N Engl J Med. 2009; 361:1045-1057.
Widimsky P, Groch L, Zelizko M, Aschermann M, Bednar F, Suryapranata H. Multicentre randomized trial comparing transport to primary angioplasty vs immediate thrombolysis vs combined strategy for patients with acute myocardial infarction presenting to a community hospital without a catheterization laboratory. The PRAGUE study. Eur Heart J 2000; 21(10):823-31.
Wong A, Mak K H, Chan C, Koh T H, Lau K W, Lim T T, Lim S T, Wong P, Sim L L, Lim Y T, et al. Combined fibrinolysis using reduced-dose alteplase plus abciximab with immediate rescue angioplasty versus primary angioplasty with adjunct use of abciximab for the treatment of acute myocardial infarction: Asia-Pacific Acute Myocardial Infarction Trial (APAMIT) pilot study. Catheter Cardiovasc Interv. 2004; 62:445-452.
Yang A, Pon Q, Lavoie A, Crawford J J, Harenberg S, Zimmermann R H, Booker J, Kelly S, Lavi S, Cantor W J, et al. Long-term pharmacodynamic effects of Ticagrelor versus Clopidogrel in fibrinolytic-treated STEMI patients undergoing early PCI. J Thromb Thrombolys. 2018; 45:225-233.

1. A method of treating acute myocardial infarction in a patient, comprising:
administering to the patient a combination of r-SAK and an antiplatelet agent prior to performing percutaneous coronary intervention on the patient.

2. The method of claim 1, wherein the acute myocardial infarction is ST-elevation myocardial infarction.

3. The method of claim 1, wherein the patient is one who is expected to receive percutaneous coronary intervention within 120 minutes from his or her first medical contact (FMC).

4. The method of claim 3, wherein the patient is one who is expected to receive percutaneous coronary intervention within 120 minutes from his or her first medical contact but more than 30 minutes from his or her first medical contact.

5. The method of claim 1, wherein the antiplatelet agent is ticagrelor.

6. The method of claim 1, wherein the r-SAK is administered intravenously.

7. The method of claim 6, wherein the r-SAK is administered in a single 5 mg bolus to the patient.

8. The method of claim 3, wherein the r-SAK is administered from about 10 min to about 60 min from the patient's FMC.

9. The method of claim 8, wherein the r-SAK is administered from about 15 min to about 60 min from the patient's FMC.

10. The method of claim 8, wherein the r-SAK is administered from about 20 min to about 50 min from the patient's FMC.

11. The method of claim 8, wherein the r-SAK is administered from about 20 min from the FMC, or about 25 min from the FMC, or about 30 min from the FMC, or about 35 min from the FMC, or about 40 min from the FMC.

12. A pharmaceutical composition for use in treating acute myocardial infarction in a patient, comprising:
r-SAK, and an antiplatelet agent.

13. The pharmaceutical combination composition of claim 12, wherein the antiplatelet agent is ticagrelor.
